(19) United States
(12) Patent Application Publication
(10) Pub. No.: US 2025/0017449 A1
(43) Pub. Date: Jan. 16, 2025

(54) MOUNTING ARM FOR A SURGICAL DEVICE

(71) Applicant: Microbot Medical Ltd., Yokneam Illit (IL)

(72) Inventors: Idan BOADER, Carmiel (IL); Simon SHARON, Zichron Yaacov (IL); Oren COHEN, Kadima (IL); Eran COHEN, Kiryat-Tivon (IL); Eyal MORAG, Tel Aviv (IL); Michael BLUMENFELD, Tel Aviv (IL); Eran ZUR, Pardes Hana Karkur (IL); Yoav TIKOCHINSKY, Tel-Aviv (IL); Rami GROSSFELD, Haifa (IL); Nir LILACH, Kfar Yehoshua (IL); Eliahu ELIACHAR, Haifa (IL)

(73) Assignee: Microbot Medical Ltd., Yokneam Illit (IL)

(21) Appl. No.: 18/713,676

(22) PCT Filed: Nov. 18, 2022
(86) PCT No.: PCT/IL2022/051231

§ 371 Date: May 27, 2024

Related U.S. Application Data
(60) Provisional application No. 63/283,633, filed on Nov. 29, 2021.

(51) Int. Cl. A61B 1/00; A61B 17/00; A61B 34/30; A61B 90/00

(52) U.S. Cl. CPC A61B 1/00149; A61B 34/30; A61B 2017/00477; A61B 2017/00991; A61B 2090/031

(57) ABSTRACT

A method for positioning a robotic device connector relative to a body access point in a patient, including:
coupling a robotic device to a movable base located at a distal end of an adjustable arm;
manipulating the adjustable arm to align a connector of the robotic device with an elongate surgical tool extending from a body access point;
linearly moving the movable base coupled to the robotic device towards the body access point to position the connector of the robotic device in proximity to a proximal end of the elongate surgical tool;
connecting the proximal end of the elongate surgical tool to the connector of the robotic device

### RELATED APPLICATION/S

This application claims the benefit of priority of U.S. Provisional Patent Application No. 63/283,633 filed on Nov. 29, 2021, the contents of which are incorporated herein by reference in their entirety.

### FIELD AND BACKGROUND OF THE INVENTION

The present invention, in some embodiments thereof, relates to alignment of a surgical device with a body access point and/or a surgical tool extended from the body access point and, more particularly, but not exclusively, to alignment of a surgical robotic device with a body access point and/or a surgical tool extended from the body access point.

Additional background art includes U.S. Patent Application Publication Number US2020/0297434A1, U.S. Pat. No. 10,549,071 B2, U.S. Pat. No. 10,744,302 B2, and U.S. Patent Application Publication Number US2018/0116736A1.

### SUMMARY OF THE INVENTION

The following describes some examples of embodiments of the invention. Some example of the invention are described herein and an embodiment may include features from more than one example and/or fewer than all features of an example:
Example 1. A method for positioning a robotic device connector relative to a body access point in a patient, comprising:
   coupling a robotic device to a movable base located at a distal end of an adjustable arm;
   manipulating the adjustable arm to align a connector of the robotic device with an elongate surgical tool extending from a body access point;
   linearly moving the movable base coupled to the robotic device towards the body access point to position the connector of the robotic device in proximity to a proximal end of the elongate surgical tool;
   connecting the proximal end of the elongate surgical tool to the connector of the robotic device.
Example 2. A method according to example 1, wherein said manipulating and said linearly moving are performed manually.
Example 3. A method according to any one of examples 1 or 2, wherein said manipulating comprises aligning said robotic device connector with a long axis of said elongates surgical tool.
Example 4. A method according to any one of the previous examples, comprising locking a position and/or orientation of said adjustable arm prior to said linearly moving.
Example 5. A method according to example 4, wherein said locking said adjustable arm in said position and/or said orientation relative to said body access point after said aligning is conducted using a torque limiting knob.
Example 6. A method according to any one of the previous examples, wherein said manipulating comprises positioning said robotic device connector at a distance shorter than 10 cm from the elongate surgical tool.
Example 7. A method according to any one of the previous examples, comprising fixing a proximal end of the adjustable arm to an anchoring point prior to said manipulating.
Example 8. A method according to any one of the previous examples, wherein a range of movement of said adjustable arm during said manipulating is at least 2 times larger than the range of movement of said movable base during said linearly moving.
Example 9. A method according to any one of the previous examples, wherein a range of movement of said adjustable arm during said manipulating has at least two additional degrees of freedom compared to the degree of movement of said movable base during said linearly moving.
Example 10. A method according to any one of the previous examples, wherein said linearly moving comprises axially moving the movable base over a maximal distance of 10 cm.
Example 11. A method according to any one of the previous examples, comprising:
   repositioning after said connecting, a distal end of the elongate surgical tool within said patient body by axially moving the robotic device relative to said movable base and along a long axis of the elongate surgical tool.
Example 12. A method according to example 11, wherein the repositioning is made by actuating a motor comprised within the robotic device.
Example 13. A method according to example 12, wherein the actuating is performed by activating the motor remotely and wirelessly using a remote-control.
Example 14. A method according to any one of examples 11 to 13, wherein said repositioning comprises repositioning said elongate surgical tool while limiting an axial range of movement of said robotic device to a maximal range of movement of 10 cm.
Example 15. A method according to any one of examples 11 to 14, comprising coupling a support member to the robotic device and to a port in said body access point and inserting at least a portion of the elongate surgical tool extending outside the patient body, into an inner space of the support member, thereby supporting the elongate surgical tool during said repositioning.
Example 16. A method according to example 15, comprising preventing buckling of said elongate surgical tool by said support member during said repositioning.
Example 17. A method according to any one of examples 15 or 16, comprising telescopically extending and/or shortening said support member during said repositioning.
Example 18. A method according to any one of examples 15 to 17, comprising prior to said coupling of the support member to the robotic device, adjusting a length of the support member in accordance with a length of the elongate surgical tool extending between the patient body and the connector of the robotic device.
Example 19. A method according to example 18, wherein adjusting the length of the support member is conducted by shortening the support member.
Example 20. A method according to example 19, wherein said shortening comprises manually cutting a body of said support member.
Example 21. A method according to example 20, wherein the manually cutting is made in one of a plurality of slits provided with the at least one portion of the support member.
Example 22. A method according to any one of examples 18 to 21, wherein the coupling a support member to the robotic device comprises coupling a proximal end of the support member to the connector of the robotic device.
Example 23. A method according to any one of the previous examples, wherein said coupling of the robotic device to the movable base comprises attaching said robotic device to said movable base using at least one snap fit connector.
Example 24. A method according to any one of the previous examples, wherein said connecting comprises connecting the proximal end of the elongate surgical tool to the connector of the robotic device to form an enclosed flow path between an inner pathway of the robotic device and an inner lumen of said elongate surgical tool.
Example 25. A method according to example 24, wherein said robotic device is used to advance at least one second elongate surgical tool into said patient body via said elongate surgical tool, and wherein said method further comprises loading the inner pathway of the robotic device with said at least one second elongate surgical tool prior to of the proximal end of the elongate surgical tool to the connector of the robotic device.
Example 26. A method according to example 25, comprising introducing a distal end of the at least one second elongate surgical tool into the inner lumen of the elongate surgical tool prior to the connecting of the proximal end of the elongate surgical tool to the connector of the robotic device.
Example 27. A method according to example 26, wherein said introducing comprises controllably introducing the at least one second elongate surgical tool via said inner lumen into said patient body using at least one second actuator of the robotic device.
Example 28. A method according to example 27, wherein the first elongate surgical tool comprises a guide-catheter and the at least one second elongate surgical tool comprises at least one of a catheter, a micro-catheter and a guidewire.
Example 29. A method according to any one of examples 27 or 28, wherein said controllably introducing is performed during a medical procedure, and wherein said method further comprises discarding said robotic device, and/or said adjustable arm after completing said medical procedure.
Example 30. A method according to any one of the previous examples, comprising:
   providing said robotic device and said adjustable arm in a single sealed sterile case.
Example 31. A method according to example 30, wherein said providing comprises providing a remote control for operating said robotic device provided in said single sealed sterile case.
Example 32. A method according to any one of the previous examples, wherein at least 80% of said robotic device and said adjustable arm is made of plastic.
Example 33. A method according to any one of the previous examples comprising, fixing a proximal end of an adjustable arm to an anchoring point.
Example 34. A maneuverable arm assembly, comprising:
   an adjustable arm configured to move in at least 3 degrees of freedom, having a distal end and a proximal end, comprising a lock configured to lock said adjustable arm in a specific orientation and/or position relative to an access point in a patient body;
   a movable base coupled to said distal end of said adjustable arm, wherein said movable base is configured to linearly move relative to said distal end, wherein said movable base comprises at least one fastener for coupling with a coupler of a robotic device.
Example 35. An arm assembly according to example 34, wherein said movable base axially slides to a maximal distance of up to 10 cm from said distal end.
Example 36. An arm assembly according to any one of examples 34 or 35, wherein a maximal extension distance of said adjustable arm between said distal end and said proximal end is in a range between 30 cm to 3 meters.
Example 37. An arm assembly according to any one of examples 34 to 36, wherein said movable base fastener is configured to reversibly couple with said robotic device coupler.
Example 38. An arm assembly according to any one of examples 34 to 37, wherein said at least one fastener comprises a snap-fit fastener.
Example 39. An arm assembly according to example 38, wherein said snap-fit fastener in said movable base comprises a protruding flexible edge shaped and sized to interlock with a snap-in region of a robotic device.
Example 40. An arm assembly according to example 39, wherein said snap-fit fastener comprising a pushable lever extending out from said movable base and functionally coupled to said protruding flexible edge, wherein pushing said pushable lever releases said protruding flexible edge from said snap-in region.
Example 41. An arm assembly according to any one of examples 34 to 40, wherein said adjustable arm lock comprises a torque limiting knob, wherein rotation of said torque limiting knob locks said adjustable arm with a limited predetermined locking force.
Example 42. An arm assembly according to any one of examples 34 to 41, wherein the distal end of said adjustable arm comprises a distal connector, and wherein said distal connector comprises a slot, and wherein said movable base is coupled to said distal connector and axially slides within said slot.
Example 43. An arm assembly according to example 42, wherein said distal connector is coupled to said distal end via a ball joint.
Example 44. An arm assembly according to any one of examples 42 or 43, wherein said movable base comprises a lock for locking said movable base at a desired position within said slot.
Example 45. An arm assembly according to example 44, wherein said movable base comprises a rotating handle with said movable base lock, wherein rotation of said handle locks said movable base in said slot.
Example 46. An arm assembly according to any one of examples 34 to 45, wherein said adjustable arm is a multi-joint arm comprising at least one first elongated portion and at least one second elongated portion pivotally connected to each other by at least one hinge passing therebetween in a central hinge portion comprising said adjustable arm lock. 25
Example 47. An arm assembly according to any one of examples 34 to 46, wherein said adjustable arm comprises a proximal connector coupled to said proximal end via a ball joint, and configured to couple said adjustable arm to an anchoring point.
Example 48. A kit, comprising:
   a maneuverable arm assembly, comprising:
   an adjustable arm configured to move in at least 3 degrees of freedom, having a distal end and a proximal end, comprising a lock configured to lock said adjustable arm in a specific orientation and/or position relative to an access point in a patient body;
   a movable base coupled to said distal end of said adjustable arm, wherein said movable base is configured to linearly move relative to said distal end, wherein said movable base comprises at least one fastener; a robotic device comprising at least one actuator and at least one exit port, wherein said robotic device is coupled to said movable base via said at least one fastener.
Example 49. A kit according to example 48, wherein said at least one fastener comprises a snap-fit connector.
Example 50. A kit according to example 49, wherein said snap-fit connector in said movable base comprises a protruding flexible edge shaped and sized to interlock with a snap-in region of said robotic device.
Example 51. A kit according to example 50, wherein said snap-fit connector comprising a pushable lever extending out from said movable base and functionally coupled to said protruding flexible edge, wherein pushing said pushable lever releases said protruding flexible edge from said snap-in region.
Example 52. A kit according to any one of examples 50 or 51, comprising an adaptor having a first end coupled to said robotic device and a second end coupled to said movable base.
Example 53. A kit according to example 52, wherein said second end of said adaptor comprises a snap-in region, and wherein said protruding flexible edge of said snap-fit connector interlocks with said snap-in region.
Example 54. A kit according to any one of examples 52 or 53, wherein said first end of said adaptor is positioned within a slot in said robotic device, and is functionally coupled to said at least one actuator of said robotic device, wherein said at least one actuator is configured to axially move said robotic device relative to said movable base by moving said adaptor within said robotic device slot.
Example 55. A kit according to example 54, wherein a maximal length of said slot is up to 15 cm.
Example 56. A kit according to any one of examples 48 to 55, comprising a control unit configured to activate said at least one actuator of said robotic device by delivering at least one signal to said robotic device.
Example 57. A kit according to any one of examples 48 to 56, comprising a telescopic support member having an inner passage along a longitudinal axis of said telescopic support member, a proximal end with a proximal connector configured to couple said proximal end to said robotic device near said at least one exit port, and a distal end with a distal connector configured to couple said distal end to a body access point, wherein said inner passage is shaped and sized to receive an elongate surgical tool extending from said body access point.
Example 58. A kit according to example 57, wherein a width of said inner passage is in a range between 2-10 mm.
Example 59. A kit according to any one of examples 57 or 58, wherein said proximal connector and/or said distal connector comprise a fastener configured to be fastened to a luer lock.
Example 60. A kit according to any one of examples 57 to 59, wherein said telescopic support member comprises at least one first movable portion and at least one second movable portion slidable within said at least one first movable portion, and wherein said at least one first movable portion and said at least one a second movable portion define said inner passage.
Example 61. A kit according to any one of examples 57 to 60, wherein activation of said at least one actuator of said robotic device axially moves said robotic device relative to said movable base with telescopically extending or shortening said telescopic support member.
Example 62. A kit according to any one of examples 57 to 61, wherein at least 80% of said support member is made of plastic.
Example 63. A kit according to any one of examples 57 to 62, wherein a maximal extension length of said telescopic support member is up to 30 cm.
Example 64. A kit according to any one of examples 57 to 63, wherein said telescopic support member is rigid.
Example 65. A kit according to any one of examples 48 to 64, wherein at least 80% of said a maneuverable arm assembly and/or said robotic device is made of plastic.
Example 66. A telescopic support member, comprising:
   an elongated telescopic body having an inner passage along a longitudinal axis of said body, a proximal end with a proximal connector configured to couple said proximal end to a robotic device connector and a distal end with a distal connector configured to couple said distal end to a body port, wherein said inner passage is shaped and sized to receive an elongate surgical tool extending from said body port.
Example 67. A telescopic support member according to example 66, wherein said body comprises at least one first movable portion and at least one second movable portion slidable within said at least one first movable portion, and wherein said at least one first movable portion and said at least one a second movable portion define said inner passage.
Example 68. A telescopic support member according to any one of examples 66 or 67, wherein a width of said inner passage is in a range between 2-10 mm.
Example 69. A telescopic support member according to any one of examples 66 to 68, wherein a maximal extension length of said telescopic support member is up to 30 cm.
Example 70. A telescopic support member according to any one of examples 66 to 69, wherein said proximal connector and/or said distal connector comprise a fastener configured to be fastened to a luer lock.
Example 71. A telescopic support member according to any one of examples 66 to 70, wherein at least 80% of said body is made of plastic.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flow chart of a process for aligning a surgical device, for example a robotic device relative to a surgical tool, e.g. a catheter, extending from a patient body, according to some exemplary embodiments of the invention;

FIG. 2A is a block diagram of a maneuverable arm assembly, for example an adjustable arm assembly, according to some exemplary embodiments of the invention;

FIG. 2B is a block diagram showing the adjustable arm assembly with a robotic device relative to an elongate surgical tool introduced in a patient, according to some exemplary embodiments of the invention;

FIG. 2C is a schematic illustration showing a control unit of the robotic device and the adjustable arm assembly with the robotic device coupled to the elongate surgical tool, according to some exemplary embodiments of the invention;

FIG. 2D is a detailed flow chart of a process for positioning a robotic device relative to a body of a patient, according to some exemplary embodiments of the invention;

FIGS. 3A-3C are schematic illustrations of an adjustable arm assembly, according to some exemplary embodiments of the invention;

FIG. 4 is a schematic illustration of a torque limiting knob of the adjustable arm, according to some exemplary embodiments of the invention;

FIGS. 5A-5B are schematic illustrations showing a robotic device attached to the adjustable arm, according to some exemplary embodiments of the invention;

FIGS. 6A-6H are schematic illustrations of a connection between the robotic device and the adjustable arm, according to some exemplary embodiments of the invention;

FIGS. 7A-7B are schematic illustrations showing the alignment of the robotic device using the adjustable arm relative to a body access point and/or an elongate surgical tool, according to some exemplary embodiments of the invention;

FIG. 8A is a schematic illustration of a robotic device attached to the deformable arm and coupled to the hollow guide,

FIG. 8B is a schematic cross-section illustration of a surgical device comprising an inner pathway aligned relative to the elongate surgical tool, according to some exemplary embodiments of the invention;

FIG. 8C is a schematic cross-section illustration zooming in on an example of an embedded Y-connector and gear assembly for rotating the elongate surgical tool, according to some exemplary embodiment of the invention;

FIGS. 8D and 8E are a schematic illustrations showing the alignment of the robotic device using the adjustable arm relative to a femoral access point (FIG. 8D), and a radial access point (FIG. 8E), according to some exemplary embodiments of the invention.

FIGS. 9A-9C are schematic illustrations of a telescopic support member, according to some exemplary embodiments of the invention; and

FIGS. 10A-10D are schematic illustrations of a telescopic support member which includes a plurality of axially aligned protrusions, according to some exemplary embodiments of the invention.

### DESCRIPTION OF SPECIFIC EMBODIMENTS OF THE INVENTION

The present invention, in some embodiments thereof, relates to alignment of a surgical device and, more particularly, but not exclusively, to alignment of a surgical robotic device.

An aspect of some embodiments relates to positioning, a robotic device, for example a surgical robotic device, relative to a body access point. In some embodiments, the robotic device is positioned relative to a surgical tool, optionally an elongate surgical tool, extending out from a patient body, for example to allow coupling between the robotic device and the surgical elongate tool. In some embodiments, positioning of the robotic device comprises aligning, for example aligning in at least one axis, for example in two or more axes, the robotic device relative to the body access point and/or relative to the surgical tool extending from the body access point. In some embodiments, the elongate surgical tool comprises a catheter, extending out from the patient body. In some embodiments, a potential advantage of aligning between a robotic device relative to the body access point and/or relative to the surgical tool extending from the body access point, is to promote guidance of the tools in the desired direction without causing unwanted movements of an elongate surgical tool inside or outside the patient. In some embodiments, the elongate surgical tool is supported by one or more mechanisms inside the robotic device, for example, by one or more wheels. In some embodiments, the elongate surgical tool is supported inside the body of the patient by the vessels in which the surgical tool is located. In some embodiments, the area that the elongate surgical tool traverses between the robotic device and the body access point and/or the surgical tool extending from the body access point should be as direct as possible in order to avoid unwanted movements of the elongate surgical tool inside the body of the patient when is being actuated, and/or to avoid buckling of the elongate surgical tool outside the patient's body instead of generating motion of the tool into the body. Therefore, in some embodiments, a potential advantage of aligning between a robotic device relative to the body access point and/or relative to the surgical tool extending from the body access point, is that unwanted movements of the elongate surgical tool inside the patient are potentially avoided.

According to some embodiments, the positioning is performed by manipulating an adjustable arm to position the robotic device at a desired distance and/or orientation relative to the elongate surgical tool. In some embodiments, the arm is manipulated in order to position an interface of the robotic device, for example a connector or an exit port of the robotic device at a desired distance and/or orientation relative to the elongate surgical tool, for example relative to a proximal end or a proximal opening of the elongate surgical tool. Alternatively or additionally, the arm is manipulated in order to position an inner pathway of the robotic device through which at least one additional elongate surgical tool is advanced towards the robotic device interface, at a desired distance and/or orientation relative to the elongate surgical tool. Optionally, the arm is used in order to align a long axis of the inner pathway of the robotic device with a long axis of the elongate surgical tool extending from the body access point. In some embodiments, the at least one additional elongate surgical tool comprises a catheter, a microcatheter and/or a guide wire. Alternatively or additionally, the additional surgical tool is a therapeutic agent, for example, embolic material, coils, beads, glue, stent retriever.

According to some embodiments, the arm is used to position the robotic device, for example the robotic device interface at a distance shorter than 20 cm, for example shorter than 20 cm, shorter than 10 cm, shorter than 5 cm or any intermediate, smaller or larger distance from the body access point, and then axially extending a portion, e.g. a distal end, of the adjustable arm to align the robotic device with a proximal end of the elongate surgical tool. Optionally, the distal end is axially extended to place the robotic device, for example the robotic device interface in contact with the elongate surgical tool, for example with a proximal end or a proximal opening of the elongate surgical tool.

According to some embodiments, the arm is used to position the robotic device at a desired distance from the body access point while moving in at least 3 degrees of freedom, for example 4 degrees of freedom, 5 degrees of freedom, 6 degrees of freedom or any larger number of degrees of freedom. In some embodiments, the arm is moved in at least two degrees of freedom more than the axially extending of the distal end of the arm.

According to some embodiments, the range of movement of said adjustable arm is at least two times larger, for example at least 3 times larger, at least 5 times larger, or any intermediate, smaller or larger value, than the axially extending of the portion of the adjustable arm. In some embodiments, the elongate surgical tool comprises at least one of a catheter, a guiding catheter, a sheath, a sleeve or a channel, extending from a body of a patient, optionally manually introduced into the patient prior to setting up the robotic device and/or prior to manipulating the arm.

According to some embodiments, after coupling the elongate surgical tool to the robotic device, the elongate surgical tool is repositioned inside the patient by moving the robotic device relative to the adjustable arm. In some embodiments, the robotic device is moved along a long axis of the elongate surgical tool, for example to advance and/or to retract the elongate surgical tool within the body. In some embodiments, the robotic device is moved using at least one actuator, for example an electric motor residing within the robotic device, and as described for example in U.S. Patent Application No. 63/195,020 filed on 30 May 2021, incorporated herein as a reference in its entirety. Optionally, the at least one actuator is activated when the robotic device receives a signal, for example a wireless signal from a control unit.

According to some embodiments, the arm is configured to allow at least two distinct types of movement. In some embodiments, a first type of movement is an “all directions” movement, characterized by a multi directional, i.e., multi-degrees-of-freedom, movement of the arm in relation to one or more of the elongate surgical tool and the body access point. In some embodiments, a second type of movement is a delicate, fine-tuning type of movement configured to perform small adjustments in the position of the robotic device in relation to one or more of the elongate surgical tool and the body access point. In some embodiments, the second type of movement is characterized by being in one orientation, i.e. having one degree of freedom, for example, forward and backward.

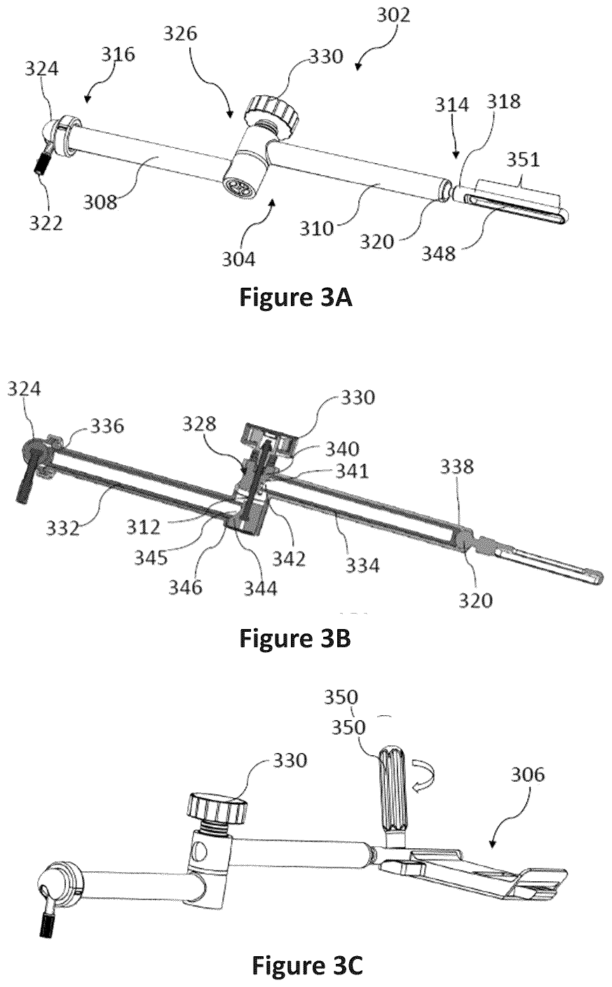

In some embodiments, once the adjustable arm is positioned at a desired location, the user can lock the position of the adjustable arm by actuating a single locking mechanism in the adjustable arm. In some embodiments, the single locking mechanism is configured to lock all adjustable locations within the adjustable arm (as will be further disclosed below, for example in relation to FIGS. 3A-C). In some embodiments, a potential advantage of providing a single locking mechanism is that it cases and shortens the time of preparation and the use of the adjustable arm for a procedure.

According to some embodiments, the robotic device is coupled to the adjustable arm via an interface, for example a first interface, functionally coupled to at least one actuator of the robotic device. In some embodiments, the first interface comprises an adaptor. Additionally or optionally, the robotic device comprises at least one second interface, for example a connector, configured to couple the robotic device to the elongate surgical tool extending from the body access point. In some embodiments, the interface between the robotic device and the arm, and/or the interface between the robotic device and the elongate surgical tool, are provided sterile and do not require the use of any drapes for any of the interface connections. In some embodiments, the coupling and/or uncoupling between the robotic device and the adjustable arm is characterized by being a quick coupling/uncoupling, which does not require the use of special tools. In some embodiments, the coupling/uncoupling between the robotic device and the adjustable arm is characterized by being a “plug and play” coupling, meaning that besides the action of coupling or uncoupling the robotic device to/from the adjustable arm, no other actions are required for the coupling/uncoupling. A potential advantage of providing such an easy coupling/uncoupling mechanisms is that, on one side, it cases and shortens the time of the preparation of the system, while on the other side, in case it is required, an easy and quick uncoupling of the robotic device from the adjustable arm can be performed.

According to some embodiments, during the repositioning of the elongate surgical tool, a support member is coupled between the robotic device and a body access point, and at least partially surrounds the elongate surgical tool to prevent buckling of the elongate surgical tool during the repositioning process. In some embodiments, the support member is a telescopic support member configured to telescopically extend or shorten during the elongate surgical tool repositioning process.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Exemplary Process for Connecting a Robotic Device to an Elongate Surgical Tool Extending from a Patient

According to some exemplary embodiments, a surgical device, for example a robotic device is used during a medical procedure. In some embodiments, the robotic device is configured to control entry of one or more tools into a patient, for example via at least one elongate surgical tool, for example, a sheath, a catheter, a guiding catheter located within the patient having a proximal end extending out from the patient body. In some embodiments, the surgical device is moved, for example manually moved, to position a connection point, for example an exit port and/or a connecting luer, of the surgical device in a desired orientation and/or position relative to a body access point, for example a puncture site, an opening in the body, for example a port in the body, or a planned location of the body opening. Alternatively or additionally, the surgical device is moved, for example manually moved, to position a connection point, for example an exit port and/or a connecting luer, of the surgical device in a desired orientation and/or position relative to a surgical tool extending from a body access point. Optionally, the desired orientation and/or position allows, for example, connecting the surgical device to a proximal end of the elongate surgical tool extending out from the body access point.

According to some exemplary embodiments, a single arm, for example a single multi-joint arm is used as a positioning device to bridge at least 80%, for example at least 85%, at least 90% or any intermediate, smaller or larger percentage value, of a distance between an anchoring point of the arm and a body access point. In some embodiments, the anchoring point is an anchoring point of the arm, and comprises a connection between the arm and at least one of a bed of a patient, a base located on a floor, and a wall mounted anchoring point. A potential advantage of using a single arm may be to minimize the footprint of positioning a device next to a bed of a patient during a surgical procedure. This allows, for example, more room for safe movement of surgical tools and/or staff next to the patient bed during the surgical process. An additional advantage of using a single arm is to enable the manipulation of the arm using only one hand, optionally keeping the other hand free to hold other devices, such as the surgical device.

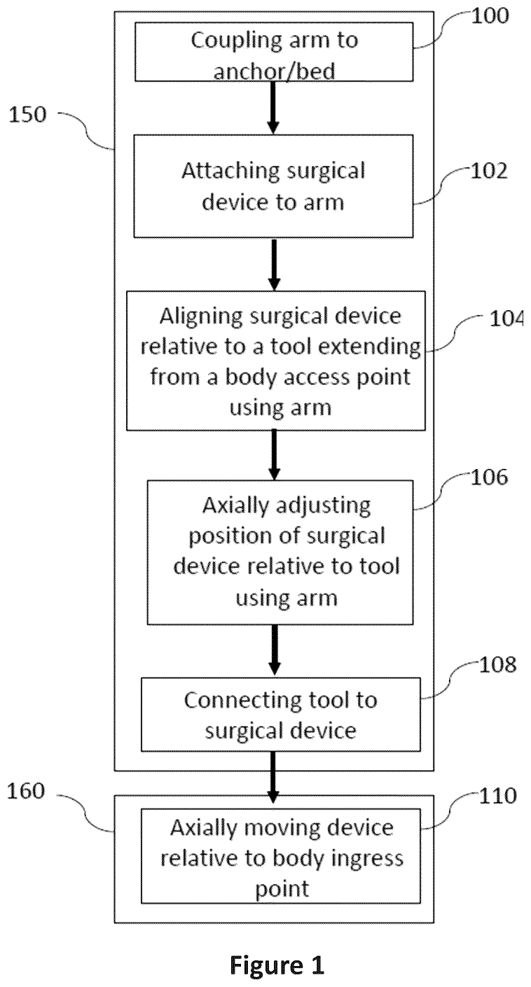

Reference is now made to FIG. 1, depicting a process for aligning a surgical device relative to an access point in a patient and/or an elongate surgical tool extending from the access point, according to some exemplary embodiments of the invention.

According to some exemplary embodiments, a proximal end of an arm, for example an adjustable arm, is coupled to an anchoring point, for example a bed of a patient, at block 100. In some embodiments, the adjustable arm is configured to move in at least 6 degrees of freedom. In some embodiments, the adjustable arm, for example a multi joint arm, allows axial extension and/or rotation of a distal end of the arm, for example an end of the arm located at a far end of the arm away from the anchoring point, close to a planned or an existing access point in the body. In some embodiments, a distal end of the arm is moved to a distance of at least 30 cm, for example at least 40 cm, at least 50 cm, at least 60 cm, at least 70 cm, or any intermediate, smaller or larger value from a proximal end of the arm connected to the bed. In some embodiments, the arm is moved to position the distal end at a distance of up to 20 cm, for example up to 15 cm, up to 10 cm, from an existing opening of the body or from a predicted location of an opening in the body. In some embodiments, the arm is configured to allow at least two distinct types of movement. In some embodiments, a first type of movement is an “all directions” movement, characterized by a multi directional movement of the arm in relation to one or more of the elongate surgical tool and the body access point. In some embodiments, this type of movement is the one performed, for example at 104.

According to some exemplary embodiments, a surgical device is coupled, for example attached, to the arm, at block 102. In some embodiments, the surgical device, for example a robotic device is coupled to the distal end of the arm, for example to a movable base located at a distal end of the arm. In some embodiments, the surgical device is coupled to the arm by a snap connector, for example a snap fastener or a snap-fit connector. Optionally, the snap connector is a snap connector and/or an adaptor integrated with the surgical device and/or the arm. In some embodiments, the surgical device is reversibly or irreversibly coupled to the arm. In some embodiments, the coupling and/or uncoupling between the robotic device and the adjustable arm is characterized by being a quick coupling/uncoupling, which does not require the use of special tools. In some embodiments, the coupling/uncoupling between the robotic device and the adjustable arm is characterized by being a “plug and play” coupling, meaning that besides the action of coupling or uncoupling the robotic device to/from the adjustable arm, no other actions are required for the coupling/uncoupling. A potential advantage of providing such an easy coupling/uncoupling mechanisms is that, on one side, it cases and shortens the time of the preparation of the system, while on the other side, in case it is required, an easy and quick uncoupling of the robotic device from the adjustable arm can be performed.

According to some exemplary embodiments, an elongate surgical tool, for example a guiding catheter, is introduced into the body through a body access point before or after the coupling of the arm, as described at block 100. Alternatively, the elongate surgical tool is introduced into the body before or after the attachment of the surgical device to the arm, described at block 102. In some embodiments, introducing the elongate surgical tool into the body comprises positioning a distal opening of the elongate surgical tool at a desired location within the body, optionally determined based on a planned surgical procedure, while a proximal opening of the elongate surgical tool is positioned outside the body. In some embodiments, the elongate surgical tool is introduced into the body via a port in a body access point. In some embodiments, the port comprises a sheath, a sleeve, a cannula.

According to some exemplary embodiments, the arm is used to align the surgical device while being coupled to the arm, relative to a surgical tool and/or a body access point, for example a body opening, at block 104. In some embodiments, the arm is manipulated to position the surgical device in a desired position and/or a desired orientation relative to the body opening location. In some embodiments, the arm moves to position the surgical device at a distance shorter than 20 cm, for example shorter than 15 cm, shorter than 10 cm, shorter than 5 cm, or any intermediate, smaller or larger distance from the body opening. In some embodiments, the arm, for example a multi-joint arm, is moved manually.

According to some exemplary embodiments, the arm is used for adjusting, for example axially adjusting a position of the surgical device relative to the body access point or to an elongate surgical tool, for example a hollow elongate surgical tool, extending out from the body access point, at block 106. In some embodiments, the position of the surgical device is axially adjusted by moving, for example linearly moving, a movable base of the arm relative to the elongate surgical tool. In some embodiments, the position of the surgical device is axially adjusted, for example to place an interface of the surgical device, such as an exit port and/or a connector, at a distance smaller than 20 cm, for example smaller than 15 cm, smaller than 10 cm, smaller than 5 cm or any intermediate, smaller or larger distance, from the tool, for example from a proximal opening of the elongate surgical tool. Optionally, the position of the surgical device is axially adjusted to place the interface of the surgical device in contact with the elongate surgical tool, for example in proximity to and/or in contact with the proximal end of the elongate surgical tool. According to some embodiments, the arm is configured to allow at least two distinct types of movement. In some embodiments, a second type of movement is a delicate type of movement configured to perform small adjustments in the position of the robotic device in relation to one or more of the elongate surgical tool and the body access point. In some embodiments, the second type of movement is characterized by being in one orientation, for example, forward and backward. In some embodiments, the second type of movement is one performed, for example, at 106.

According to some exemplary embodiments, the tool, for example the elongate surgical tool, is connected to the surgical device, at block 108. In some embodiments, the proximal end of the elongate surgical tool is connected to a distal opening and/or an exit port, and/or a connector of the surgical device, for example to generate a flow path between the inner pathway of the surgical device and a lumen of the elongate surgical tool.

According to some exemplary embodiments, the surgical device is moved relative to the body access point at block 110. In some embodiments, the surgical device axially moves, for example using at least one actuator, e.g. motor, of the surgical device, relative to the body access point. In some embodiments, the movement of the robotic device repositions the connected elongate surgical tool within the body, for example advances or retracts the distal end of the elongate surgical tool inside the patient's body.

In some embodiments, block 150, that includes the sequences described in blocks 100, 102, 104, 106 and 108, is conducted during the setup of the surgical device, prior to conducting the medical procedure. In some embodiments, block 160, which includes the sequence detailed in block 110, is conducted during the medical procedure.

### Exemplary Adjustable Arm Assembly

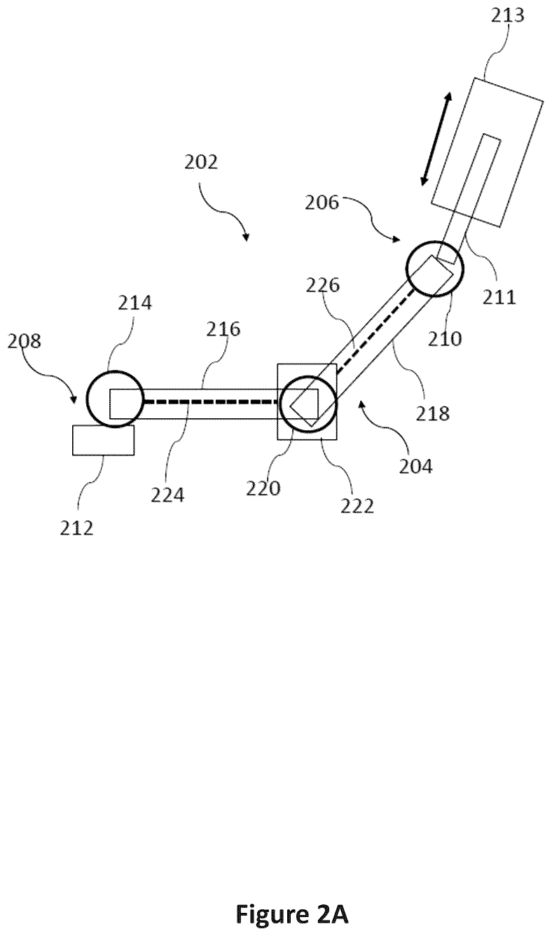

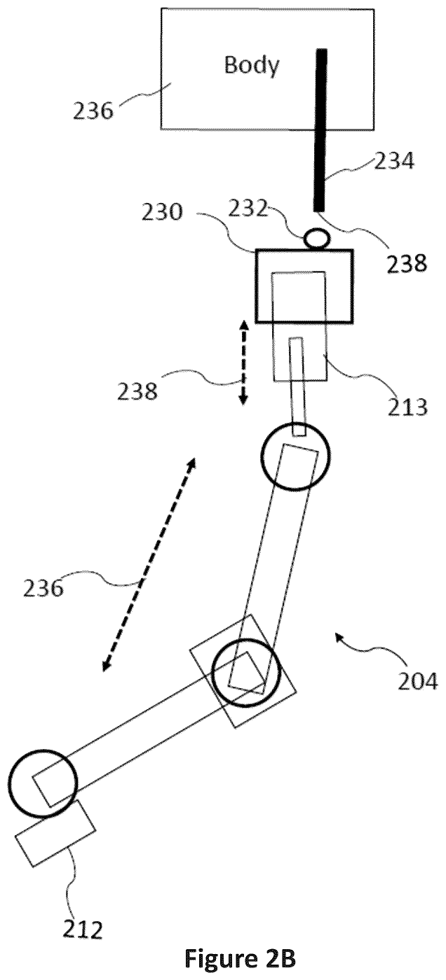

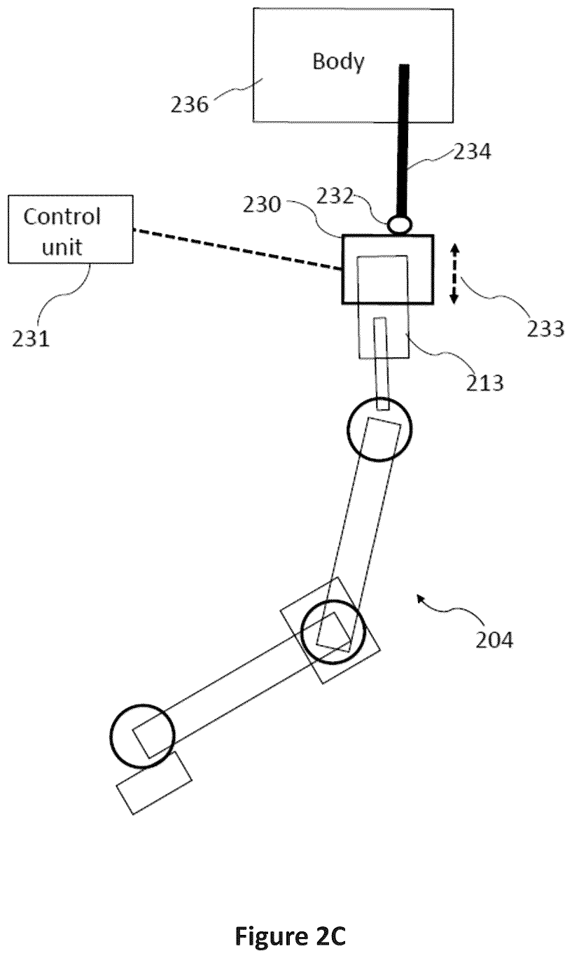

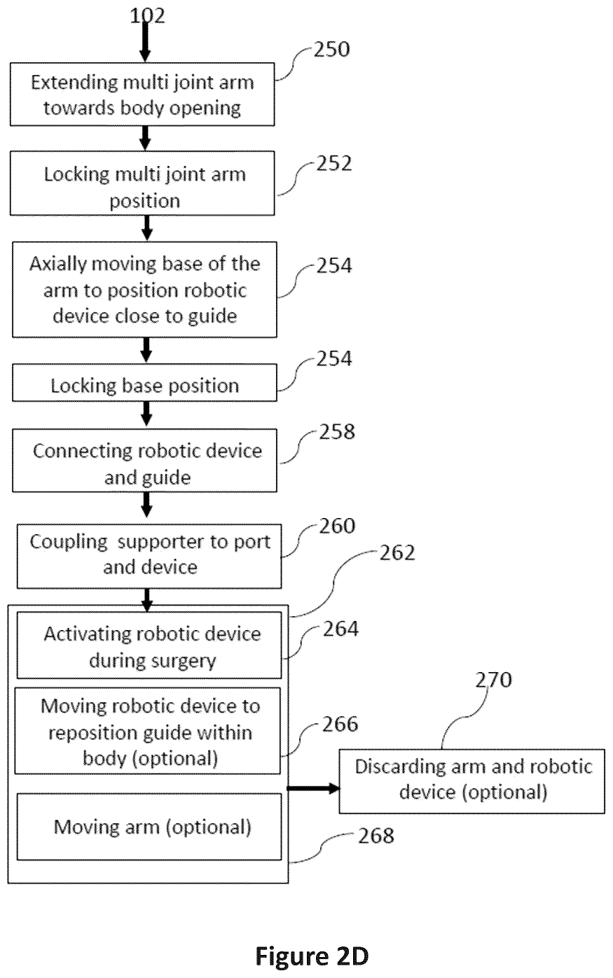

Reference is now made to FIG. 2A, depicting an adjustable arm assembly, according to some exemplary embodiments of the invention.

According to some exemplary embodiments, an adjustable arm assembly, for example assembly 202 comprises an elongated body 204 having a distal end 206 and a proximal end 208. In some embodiments, the body 204 comprises a distal connector 211 at a distal end of the arm body 204. In some embodiments, the distal connector is coupled to the body 204 by at least one joint, for example a ball joint 210. In some embodiments, the distal connector 211 is configured to be functionally coupled to an adjustable base 213 which is shaped and sized to receive a robotic device.

According to some exemplary embodiments, the body 204 comprises at least one proximal connector 212 at the proximal end 208. In some embodiments, the proximal connector 212, optionally comprising a clamp, is configured to attach the arm assembly 202 to an external base, for example a bed of a patient or to a bed in an operating room. Alternatively, the proximal connector is configured to connect the arm assembly 202 to an anchor, for example a table, a cart, or a wall mounted anchor. In some embodiments, the proximal connector 212 is coupled to the body 204 by at least one joint, for example a ball joint 214.

According to some exemplary embodiments, the body 204 comprises at least one first elongated portion 216, and at least one second elongated portion 218, coupled to each other by a connector 220. In some embodiments, the at least one first elongated portion 216 is coupled to the connector 212 via the joint 214. In some embodiments, the at least one second elongated portion 218 is coupled to the distal connector 211 via joint 210. In some embodiments, the connector 220 comprises a pivot, and the at least one first elongated portion 216, and the at least one second elongated portion 218 are pivotally coupled to each other by the pivot 220. Alternatively, the connector 220 comprises a joint, for example a ball joint.

According to some exemplary embodiments, the body 204 comprises at least one lock, for example a central lock 222, configured to lock the at least one first elongated portion 216 and the at least one second elongated portion 218 in a specific position and/or orientation relative to each other. Additionally or optionally, the central lock 222 locks the at least one first elongated portion 216 at a specific position and/or orientation relative to the connector 212, for example by applying force against the joint 214. Alternatively or optionally, the central lock 222 locks the at least one second elongated portion 218 in a specific position and/or orientation relative to the distal connector 211, for example by applying force against the joint 214.

According to some exemplary embodiments, the central lock 222 comprises at least one shaft 224 coupled to the at least one first elongated portion 216, and connecting the central lock 222 and the joint 214. Alternatively or additionally, the central lock 222 comprises at least one additional shaft 226 coupled to the at least one second elongated portion 218, and connecting the central lock 222 and the joint 210. In some embodiments, the central lock 222, comprises a force limiting lock, for example a force limiting rotary lock. In some embodiments, rotation of the central lock axially moves each shaft of shafts 224 and 226, and pushes an end of each shaft against the ball joints, for example ball joints 214 and 210, respectively. The force applied by each shaft on the ball joint prevents the movement of the ball joint. Optionally, the force limiting lock comprises a torque limiting knob.

According to some exemplary embodiments, the assembly 202 comprises at least one movable base 213 configured to be coupled to a housing of a robotic device. In some embodiments, the movable base 213 is attached to the distal connector 211. In some embodiments, the distal connector 211 comprises a slot, for example am linear slot configured to allow axial movement of the base 213 within the slot, relative to the connector 211.

According to some exemplary embodiments, the movable base 213 is attached to the distal connector using at least one snap fastener, for example a snap-fit connector, which allows for example reversibly attachment of the movable base 213 to the arm body 204. In some embodiments, the movable base 213 comprises at least one aperture that is shaped and sized to receive a protrusion extending from a robotic device housing, for example to allow coupling of the robotic device to the movable base.

According to some exemplary embodiments, the movable base 213 or the distal connector 211 comprise at least one movable base lock, for locking the movable base at a specific axial position relative to the distal connector 211 or the arm body 204.

According to some exemplary embodiments, for example as shown in FIG. 2B, the arm assembly 204 is used to align a surgical device 230, for example an opening 232 of the surgical device 230, with a elongate surgical tool 234, extending out from a body 236 of a patient. In some embodiments, the arm assembly 204 is used to align the opening 232 with a proximal opening 238 of the elongate surgical tool 234.

According to some exemplary embodiments, the arm assembly 204 is used to bring an opening 232 of the surgical device 230 to a distance from an opening 238 of a elongate surgical tool 234 which is short enough to allow connection between the opening 232 and the opening 238, to generate a stable flow path between the surgical device 230 and the body 236. In some embodiments, the arm, which is optionally a single, multi-joint arm is moved in at least two axially movements, a first long range axial movement and a second short range axial movement. In some embodiments, the first long range axial movement 236 is larger than 50 cm, for example larger than 60 cm, larger than 70 cm, larger than 80 cm, larger than 100 cm or any intermediate, smaller or larger value. In some embodiments, the second short range axial movement 238 of the arm assembly is shorter than 40 cm, for example shorter than 30 cm, shorter than 20 cm, shorter than 10 cm, shorter than 5 cm or any intermediate, smaller or larger value.

According to some exemplary embodiments, once the opening 232 is aligned with the opening of the elongate surgical tool, the robotic device is connected to the elongate surgical tool 234, for example as shown in FIG. 2C. In some embodiments, for example during a surgical procedure, the elongate surgical tool is repositioned within the body 236 by moving, for example axially moving the robotic device 230 relative to movable base 213. In some embodiments, the robotic device 230 is axially moved using at least one actuator, for example an electric motor of the robotic device. In some embodiments, the robotic device axially moves in response to a signal received from a control unit 231. Optionally, the control unit 231 transmits a signal, for example a wireless signal that activates the motor in the robotic device used for the axial movement 233.

According to some exemplary embodiments, the control unit 231, the arm assembly 204, and the robotic unit 230 are provided as a kit, optionally in at least one sealed case, for example within at least one sealed plastic bag. Optionally, at least one first interface, for example an adaptor, for coupling the robotic device to the arm assembly and at least one second interface, for example a connector for coupling the robotic device to the elongate surgical tool, are not provided within a scaling case. In some embodiments, at least 60%, for example at least 70%, at least 80%, at least 90% or any intermediate, smaller or larger percentage value, of the arm assembly 204, the control unit 231 and/or the robotic device 230 is formed from plastic.

According to some embodiments, the robotic device is a small robotic device having a weight smaller than 500 grams (gr), for example smaller than 400 gr, smaller than 300 gr, smaller than 200 gr or any intermediate, smaller or larger value. In some embodiments, the robotic device comprises at least one actuator, for example an electric motor, for advancing a micro-catheter into the elongate surgical tool. Optionally, the robotic device comprises at least one additional electric motor, for moving the robotic device relative to the at least one adjustable base of the arm.

### Exemplary Detailed Process for Positioning a Robotic Device Relative to a Body

According to some exemplary embodiments, a robotic device, optionally used during a surgical procedure is positioned at a specific fixed location and/or orientation relative to a body of the patient, for example a patient undergoing the surgical procedure. In some embodiments, the robotic device is positioned at a fixed location and/or orientation relative to a elongate surgical tool extending from the body opening, for example to establish a flow path between the robotic device and the body via the elongate surgical tool.

According to some exemplary embodiments, the positioning of the robotic device and/or any adjustment of the positioning is performed using movements of a maneuverable arm, for example a single multi-joint maneuverable arm and/or by short range movements of the robotic device, in order to minimize a foot print of a positioning system next to a patient bed during surgery. In some embodiments, minimizing the foot print may allow easier access of surgical tools and/or staff to the patient bed during the surgical process. Reference is now made to FIG. 2D, depicting a process for positioning of a robotic device relative to a patient body, for example relative to an access point, for example an opening, in a patient body, before and/or during a surgical procedure, according to some exemplary embodiments of the invention.

According to some exemplary embodiments, after the attachment of the surgical device, for example robotic device to the arm at block 102, the arm is moved, for example, extended towards the body opening, at block 250. In some embodiments, the arm, for example a multi-joint arm is extended by moving at least two elongated portions of the arm relative to each other. In some embodiments, a body of the arm, for example body 204 shown in FIG. 2A, is configured to extend to a maximal distance of up to 2 meters, for example up to 1.7 meters, up to 1.5 meters, up to 1.3 meters, up to 0.5 meter or any intermediate, smaller or larger distance between a proximal end of the arm, for example proximal end 208 to a distal end of the arm, for example distal end 206.

According to some exemplary embodiments, a position of the arm is locked at block 252. In some embodiments, a position of the arm is locked using a single lock, for example a force limiting lock, for example lock 222 shown in FIG. 2A. In some embodiments, locking of the arm position at block 252 comprises locking the at least two elongated portions of the arm, pivotally connected to each other, at a fixed angle therebetween.

According to some exemplary embodiments, a base of the arm coupled to the robotic device is axially moved, for example linearly moved, at block 254. In some embodiments, the base, for example base 213 shown in FIG. 2A is linearly moved relative to the arm body 204, to position the robotic device closer to an elongate surgical tool, extending out from the body opening. Alternatively or additionally, the base axially moves to align at least one opening of the robotic device with a proximal opening of the elongate surgical tool. In some embodiments, a maximal axial extension of the base relative to a distal end 206 of the arm is up to 30 cm, for example up to 20 cm, up to 10 cm, or any intermediate, smaller or larger value. In some embodiments, the base is axially moved to position an opening of the robotic device at a distance smaller than 40 cm, for example smaller than 30 cm, smaller than 20 cm, smaller than 10 cm, smaller than 5 cm from the elongate surgical tool opening.

According to some exemplary embodiments, the base is locked at a specific position, at block 254, for example to prevent further axial movement of the base.

According to some exemplary embodiments, the robotic device and the elongate surgical tool are connected, at block 258. In some embodiments, the elongate surgical tool, for example a guiding catheter or sheath is connected to an opening of the robotic device, for example to form a flow path between the robotic device and a distal opening of the elongate surgical tool positioned within the patient body. In some embodiments, a proximal connector of the elongate surgical tool located near or surrounding the elongate surgical tool proximal opening is connected to a connector at the robotic device opening. Optionally, a section of the elongate surgical tool extending out from the body opening has a length of up to 50 cm, for example up to 40 cm, up to 30 cm, up to 20 cm or any intermediate, smaller or larger value.

According to some exemplary embodiments, the elongate surgical tool is connected to the robotic device opening without moving a distal end of the hollow device located within the patient body, or with moving the distal end to a distance of less than 10 cm, for example less than 5 cm, less than 3 cm within the patient body.

According to some exemplary embodiments, at least one guide supporter is coupled to a port in the body opening through which the elongate surgical tool penetrates into the body, and to the robotic device, at block 260. In some embodiments, the at least one guide supporter surrounds, at least partly, the elongate surgical tool between the body opening and the robotic device. Optionally, the at least one guide supporter is coupled to an external surface of a connector of the robotic device to which the elongate surgical tool is connected. In some embodiments, the at least one guide supporters, supports the elongate surgical tool to prevent kinks or bending of the elongate surgical tool, when the elongate surgical tool is repositioned within the patient body by movements of the robotic device.

According to some exemplary embodiments, during a surgical procedure 262, the robotic device is activated to introduce at least one surgical device into the body of the patient via the elongate surgical tool, at block 264. In some embodiments, at least one actuator of the robotic device, for example, at least one electric motor is activated at block 264. In some embodiments, the at least one actuator of the robotic device is activated and/or controlled using a control unit, for example a remote control unit.

According to some exemplary embodiments, during the surgical procedure 262, the robotic device is optionally moved in order to reposition the elongate surgical tool within the body, at block 266. In some embodiments, the robotic device axially advances towards and/or backwards from the body opening, while being connected to the elongate surgical tool, to reposition a distal end of the elongate surgical tool within the body. In some embodiments, the robotic device is moved using at least one additional actuator within the robotic device, translating energy to an axial movement of the robotic device relative to the arm, for example relative to a movable base of the arm to which the robotic device is attached.

According to some exemplary embodiments, the arm is optionally moved during the surgical procedure, at block 268. In some embodiments, the arm is moved by releasing one or more locks, for example to reposition the robotic device relative to the body opening and/or the elongate surgical tool.

According to some exemplary embodiments, once the surgical procedure 262 ends, the arm and the robotic device are discarded. Optionally or additionally, the control unit used, for example, to activate at least one actuator of the robotic device is also discarded after the surgical procedure ends.

### Exemplary Maneuverable Arm

Reference is now made to FIGS. 3A-3C depicting a maneuverable arm assembly, according to some exemplary embodiments of the invention.

According to some exemplary embodiments, a maneuverable arm assembly, for example assembly 302 comprises an adjustable arm 304 and a movable base 306, shown in FIG. 3C, coupled to the adjustable arm 304.

According to some exemplary embodiments, the adjustable arm 304 is a multi-joint arm, comprising at least one first elongated portion 308 and at least one second elongated portion 310, connected, for example pivotally connected, to each other by at least one hinge 312. In some embodiments, each of the at least one first elongated portion 308 and at least one second elongated portion 310 comprise a ring-shaped portion. In some embodiments, the ring-shaped portions are axially positioned adjacent to each other, and are shaped and sized to receive the hinge 312 to form a central cylindrical hinge portion 326, pivotally connecting the two elongated portions, 308 and 310, of the arm 304.

According to some exemplary embodiments, the arm 304 has a distal end 314 and a proximal end 316. In some embodiments, for example as shown in FIGS. 3A and 3B, the arm 304 comprises a distal connector 318 at the distal end 314 coupled to the at least one second elongated portion 310, by a joint 320, for example a ball joint.

According to some exemplary embodiments, the arm 304 comprises a proximal connector 322 for connecting the arm 304 to at least one anchor, for example an anchor positioned on a floor, a bed of a patient, a cart or a wall-mounted anchor. In some embodiments, the proximal connector 322 is coupled to the at least one first elongated portion 308 by a joint 324, for example a ball joint.

According to some exemplary embodiments, the arm 304 comprises at least one lock for example a central lock 328 positioned within the central cylindrical hinge portion 326. In some embodiments, the central lock comprises a rotary lock, optionally a force limiting rotary lock. In some embodiments, the central lock 328 comprises a central shaft, which is optionally the hinge 312 passing through the central cylindrical hinge portion 326, and is connected to an external knob 330, located outside the hinge portion 326.

According to some exemplary embodiments, the central lock 328 comprises at least one shaft 332 passing within the at least one first elongated portion 308, connecting the joint 324 and the central cylindrical hinge portion 326. In some embodiments, a distal end 336 of the shaft 332 faces the joint 324, optionally has a concaved shape, shaped and sized to match at least partly an external curvature of the joint 324. Additionally, the central lock 328 comprises at least one additional shaft 334 passing within the at least one second elongated portion 310, connecting the joint 320 and the central cylindrical hinge portion 326. In some embodiments, a distal end 338 of the shaft 334 faces the joint 320, optionally has a concaved shape, shaped and sized to match at least partly an external curvature of the joint 320.

According to some exemplary embodiments, the central lock 328 comprises at least one rotating member 340 coupled to the shaft 312, facing a proximal end 342 of the shaft 334, and at least one additional rotating member 344 coupled to the shaft 312, facing a proximal end 346. In some embodiments, each of the rotating members 340 and 344 comprises a cutaway section, for example cutaway sections 341 and 345, respectively.

According to some exemplary embodiments, the central lock 328 is configured to move between a first state in which the elongated portions 308 and 310 can move freely relative to each other, and a second state in which the elongated portions 308 and 310 are locked in a specific position relative to each other. In some embodiments, the central lock 328 moves between the first open state and the second lock state, when the external knob 330 is rotated. In some embodiments, rotation of the knob 330 is translated by rotation of the shaft 312 and rotation, for example simultaneous rotation of each of the rotating members 340 and 344, to an axial movement, for example a simultaneous axial movement, of shaft 332 towards joint 324, and shaft 334 towards joint 320.

According to some exemplary embodiments, in the first open state, a cut away section of each of the rotating members 340 and 344 faces the proximal ends 342 and 346. In some embodiments, rotation of the external knob 330 rotates the rotating members 340, and pushes a solid section of the rotating member 340 against the shaft 334, pushing the distal end 338 against the joint 320. Additionally and optionally simultaneously, rotating members 344 also rotates, and pushes a solid section of the rotating member 344 against the shaft 332, pushing the distal end 336 against the joint 324.

Alternatively, in some embodiment, rotation of the central shaft pulls each of the rotating members 340 and 344 upwardly towards the knob 330, causing the cut away sections of each rotating member to push sideways each of the shafts 332 and 334, towards joints 324 and 320 respectively.

According to some exemplary embodiments, the force applied by each distal end 336 and 338 on the joints 324 and 320, respectively, prevents further movement of the joints and/or further movement of each of the elongated portions 308 and 310 relative to each other.

According to some exemplary embodiments, a distal connector 318 of the arm 304 comprises at least one slot 348. In some embodiments, the movable base 306 is attached to the distal connector 318 and is configured to axially move, for example axially slide within the slot 348. In some embodiments, a length 351 of the slot determines a range of axial movement of the movable base, used for example as described at blocks 106 and 254, to position a robotic device attached to the movable base closer to a body opening or to a elongate surgical tool extending from the body opening. In some embodiments, a maximal length 351 of the slot 348 is in a range between 5 cm-30 cm, for example 5 cm-15 cm, 10 cm-30 cm or any intermediate, smaller or larger range of values.

According to some exemplary embodiments, the movable base 306 comprises a handle 350 coupled to the movable base through slot 348, and is used to move the movable base within the slot 348. In some embodiments, the handle 350 has a round cylindrical shape with a plurality of protrusions outwardly extending from the external surface of the handle 350, for example to allow easy gripping of the handle 350 with a single hand. In some embodiments, the handle 350 comprises at least one movable base lock for locking the movable base 306 in a desired position within the slot 348. In some embodiments, the at least one movable base lock comprises a friction lock, where rotation of the handle 350 applies force against the distal connector 318 which increases the friction between the handle 350 and/or the movable base 306 and the distal connector 318. In some embodiments, the increased friction locks the movable base within the slot 348.

In some embodiments, actuation of knob 330 causes the locking of all adjustable parts in the adjustable arm. For example, closing knob 330 causes the locking of the movable base 306, the at least one first elongated portion 308 and at least one second elongated portion 310, connected, for example pivotally connected, to each other by at least one hinge 312. In some embodiments, a potential advantage of providing a “one-knob-close-all” mechanism is that it cases the use of the adjustable arm and potentially shortens the time required to prepare the system in a procedure.

### Exemplary Torque Limiting Knob

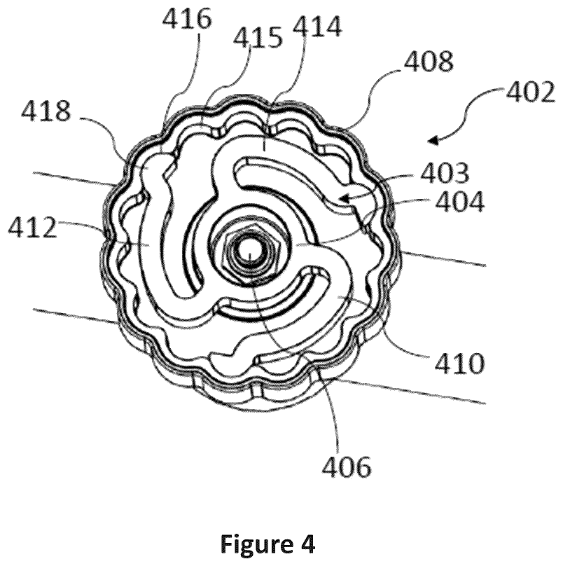

According to some exemplary embodiments, the arm comprises at least one torque limiting knob, for example to allow locking of the arm movement while applying a limited amount of torque. Reference is now made to FIG. 4 depicting a torque limiting knob, according to some exemplary embodiments of the invention.

According to some exemplary embodiments, a torque limiting knob, for example knob 402 comprises an inner rotating member 403 coupled to a central perpendicular shaft 406, and an outer rotating member 408, optionally a ring-shaped rotating member. In some embodiments, the inner rotating member is coaxially positioned within the outer rotating member 408. In some embodiments, the inner rotating member 403 comprises a ring 404 and at least one, for example a plurality, of elastic protrusions, for example protrusions 410 and 412, outwardly extending from the ring 404. In some embodiments, each of the clastic protrusions is angled and has a free end that is pressed against the inner surface of the outer rotating member 408.

According to some exemplary embodiments, the inner surface of the outer rotating member 408 comprises a plurality of concave sockets, for example sockets 415 and 416, distributed along the inner circumference of the outer rotating member 408, facing the inner rotating member 403. In some embodiments, the free end of each clastic extension, for example free end 418, has a convex-shaped tip shaped and sized to fit inside each of the sockets 415 and 416.

According to some exemplary embodiments, the torque limiting knob is the external knob 330 which is part of the central lock 328 shown in FIG. 3B. In some embodiments, rotation of the outer rotating member 408 rotates the inner rotating member 404 and the shaft 406 to close the central lock from an open state to a lock state. In some embodiments, the rotation of the outer rotating member 408 is translated to rotation of the inner rotating member based on a friction between the convex-shaped tip and the concave sockets, and a force resisting the turning of the shaft 406. In some embodiments, as long as the turning resisting force, for example a locking force of the central lock, is smaller than a force generating the friction between the convex-shaped tip and the concave sockets, the rotation of the outer rotating member 408 is translated to rotation of the inner rotating member 403. In some embodiments, when the turning resisting force applied on the shaft, for example the locking force, is higher than the force generating the friction, the outer rotation member slides over the elastic protrusions and moves relative to the inner rotating member 403.

### Exemplary Mounting a Robotic Device on the Arm Assembly

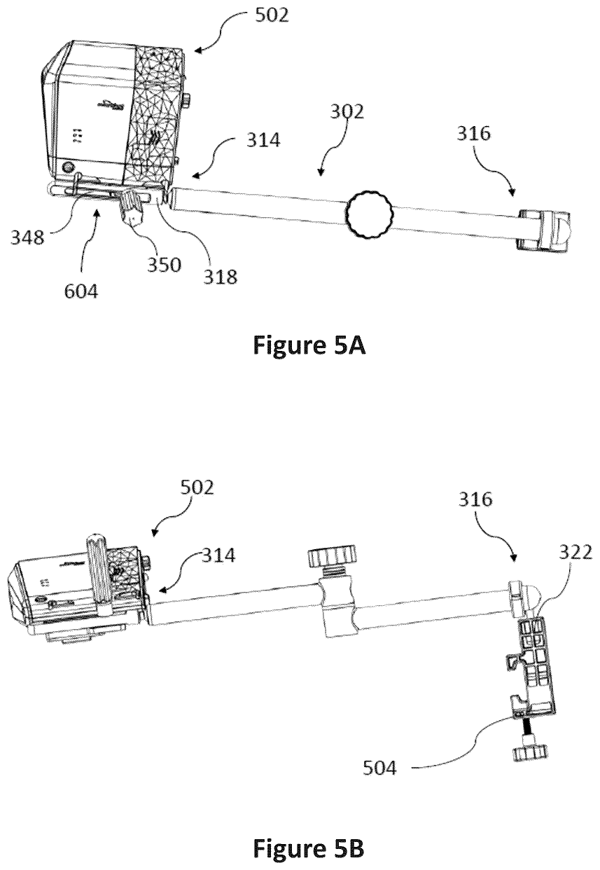

Reference is now made to FIGS. 5A and 5B, depicting a robotic device mounted on a maneuverable arm, according to some exemplary embodiments of the invention. According to some exemplary embodiments, a robotic device 502 is mounted on a movable base 604 at a distal end 314 of arm assembly 302. In some embodiments, a proximal connector 322 of the arm assembly 302 is coupled to a clamp 504, configured, for example, to couple the arm assembly 302 to a bed of the patient. In some embodiments, the robotic device 502 has a thin profile housing, and is optionally connected lengthwise to the movable base of the arm assembly 302.

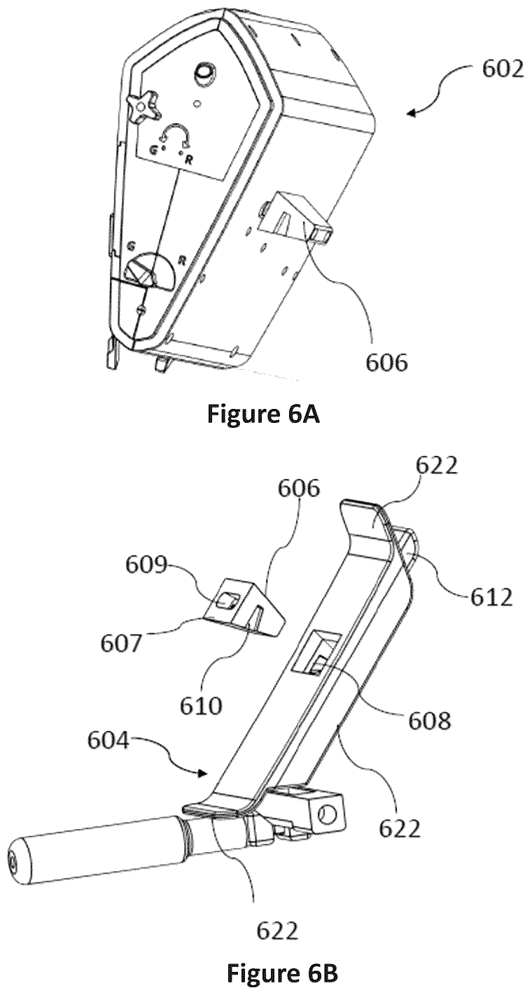

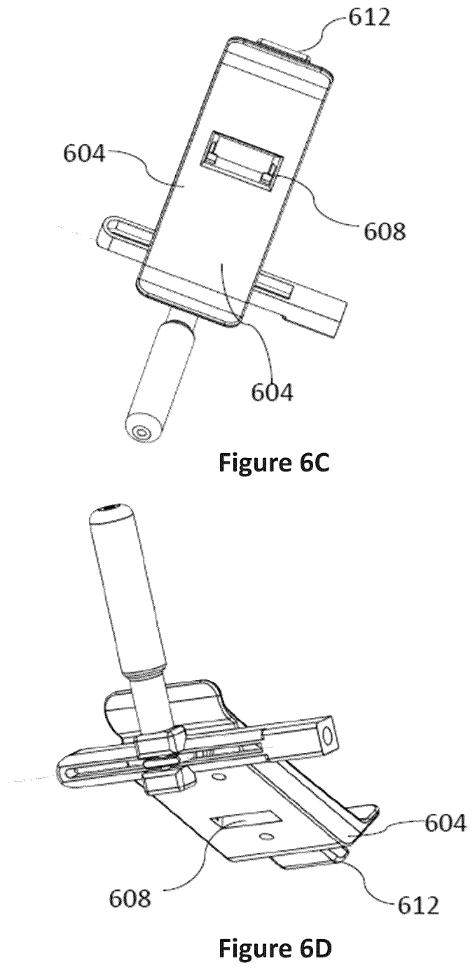

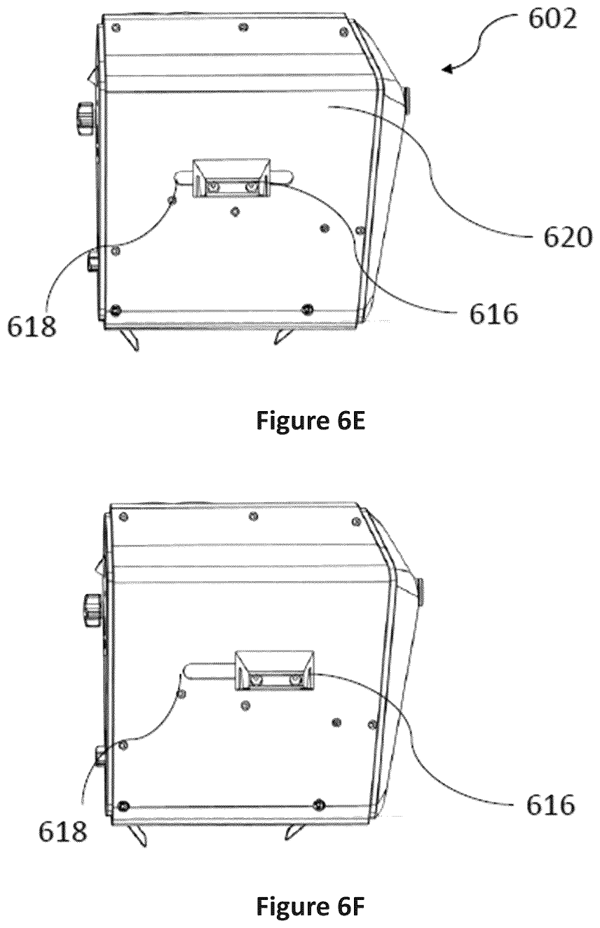

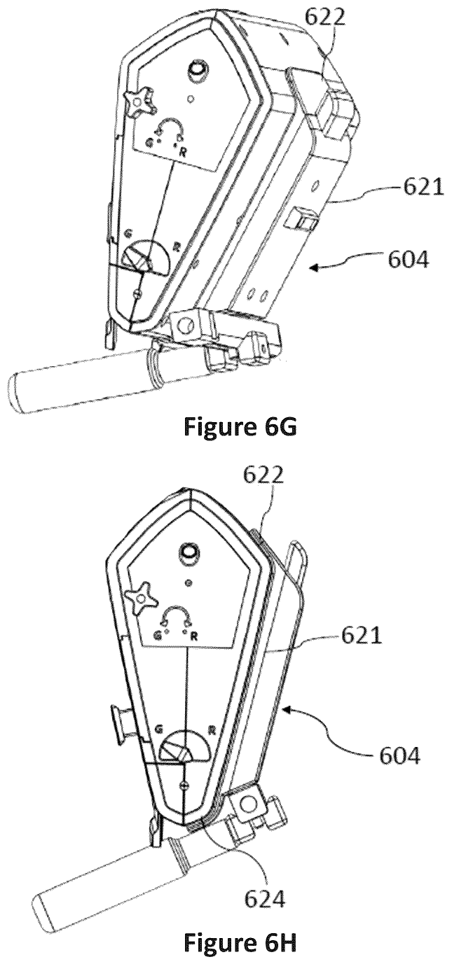

Reference is now made to FIGS. 6A-6H, depicting fastening of the robotic device to the movable base, according to some exemplary embodiments of the invention.

In some embodiments, as mentioned above, the coupling and/or uncoupling of the robotic device and the movable base of the adjustable arm is characterized by being a quick coupling/uncoupling, which does not require the use of special tools. In some embodiments, the coupling/uncoupling of the robotic device and the movable base of the adjustable arm is characterized by being a “plug and play” coupling, meaning that besides the action of coupling or uncoupling the robotic device to/from the adjustable arm, no other actions are required for the coupling/uncoupling. A potential advantage of providing such an easy coupling/uncoupling mechanisms is that, on one side, it cases and shortens the time of the preparation of the system, while on the other side, in case it is required, an easy and quick uncoupling of the robotic device from the adjustable arm can be performed. The mechanism behind the easy coupling/uncoupling are described, for example—and not only, in the following paragraphs in relation to adaptor 606 and one aperture 608.

According to some exemplary embodiments, the robotic device 602 is coupled to the movable base 604 using at least one adaptor 606. In some embodiments, a first end 607 of the adaptor 606 is shaped and sized to penetrate and interlock with the robotic device housing, and opposite end of the adaptor 606 is shaped and sized to penetrate into at least one aperture 608 in the movable base 604, and to interlock with a fastener, for example a snap fit connector of the movable base 604. In some embodiments, at least one to flexible and/or a movable protrusion of a snap-fit connector of the movable base is positioned within the at least one aperture 608, and is configured to deflect and/or translate and penetrate into an aperture, for example a side aperture 610 of the adaptor 606, when the adaptor 610 is pushed into the aperture 608 and deflects and/or pushes the flexible and/or movable protrusion, optionally against at least one spring located within the housing of movable base 604. In some embodiments, penetration of the flexible and/or movable protrusion into the side aperture 610 interlocks the adaptor 606 to the movable base 604. Optionally the protrusion is held in place against the aperture 610 by the at least one spring.

According to some exemplary embodiments, the snap fit connector comprises a release shaft functionally coupled to the at least one flexible/movable protrusion. In some embodiments, the release shaft, or at least an end 612 of the release shaft extends out from the movable base. In some embodiments, pressing the release shaft end 612 deflects the at least one flexible protrusion, pushing the flexible protrusion out from the side aperture 610, which allows, for example release of the adaptor 606 from the movable base 604. Alternatively or additionally, pressing the release shaft end 612 pushes the at least one movable protrusion against the at least one spring, thereby pushing the protrusion out from the side aperture 610, which allows, for example release of the adaptor 606 from the movable base 604.

According to some exemplary embodiments, for example as shown in FIG. 6B, the first end 607 of the adaptor 606 is shaped and sized to be connected to the robotic device 602. In some embodiments, the adaptor 606 is configured to be connected to a connector 616 of the robotic device, shown in FIGS. 6E and 6F. In some embodiments, the connector 616 is a sliding connector, configured to controllably slide within a recess optionally a slot, for example a linear slot 618 in a housing 620 of the robotic device 602. In some embodiments, for example as shown in FIG. 6F, the sliding connector 616 is configured to axially slide within slot 618. In some embodiments, the sliding connector 616 is functionally connected to at least one actuator, for example an electric motor configured to controllably move the sliding connector 616 within the slot 618. In some embodiments, a sliding range is between 0 and 30 cm, for example between 0 and 20 cm, between 0 and 10 cm or between 0 and 5 cm, or any intermediate, smaller or larger sliding range.

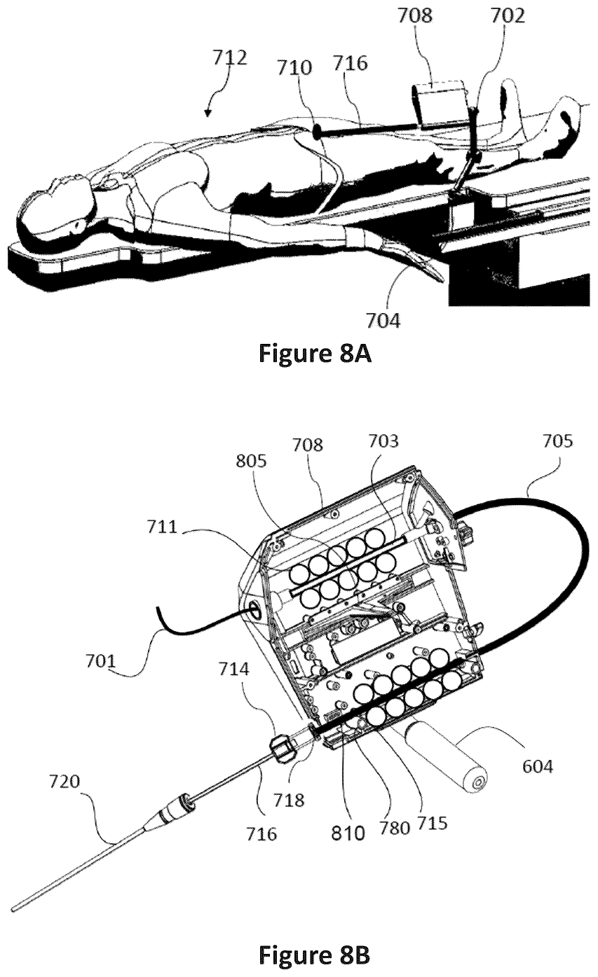

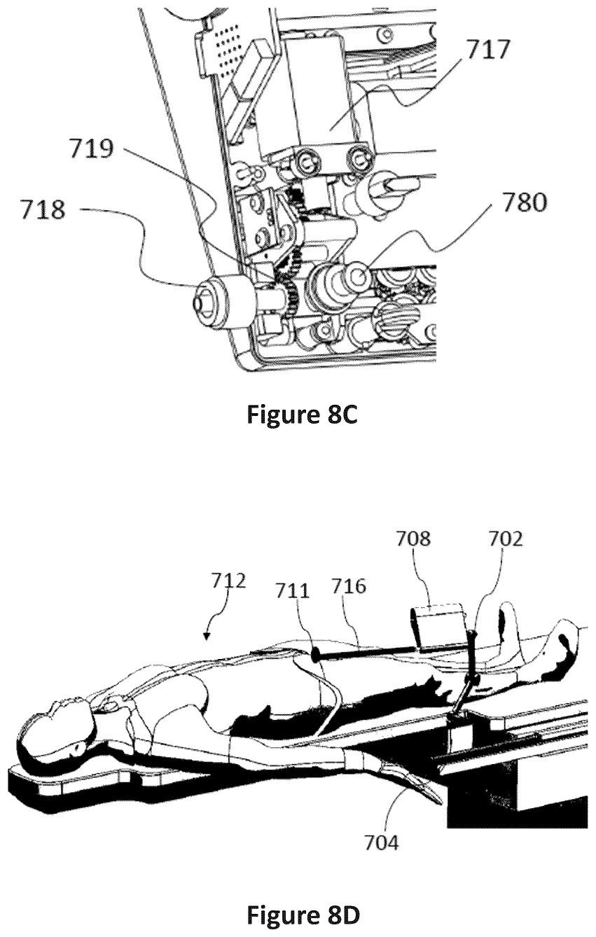

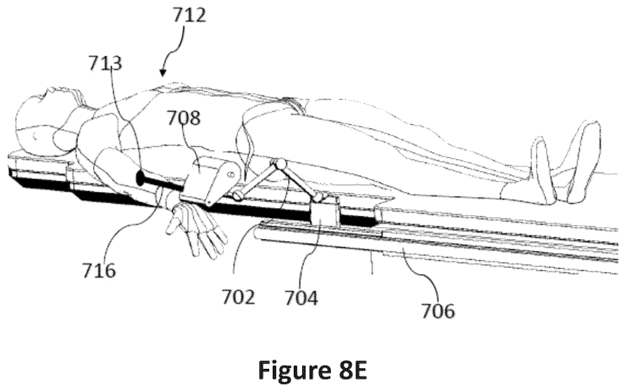

According to some exemplary embodiments, the sliding connector 616 comprises an aperture which is shaped and sized to receive the first end 607 of the adaptor 606 or a protrusion at the first end. Alternatively or additionally, the sliding connector comprises at least one protrusion which is shaped and sized to fit into an aperture 609 at the first end 607. In some embodiments, the adaptor 606 interlocks, optionally reversibly interlocks, with the sliding connector 616. Optionally, the adaptor is released from the sliding connector 616 using at least one actuator of the robotic device. Alternatively, the sliding connector 616 comprises a snap fastener, for example a snap-fit connector that allows, reversibly coupling of the adaptor 606 to the sliding connector 616. Alternatively, the adaptor is irreversibly coupled to the sliding connector 616. In some embodiments, the connector 616 is functionally coupled to a sliding member 805 of the robotic device 708 shown in FIG. 8B. In some embodiments, the sliding member 805 is functionally connected to at least one actuator of the robotic device 708 configured to move the sliding member 805 for controllably sliding within the slot, for example as described in U.S. Patent Application No. 63/195,020 filed on 30 May 2021, incorporated herein as a reference in its entirety.

According to some exemplary embodiments, for example as shown in FIGS. 6B, 6G and 6H the movable base 604 comprises a base and at least one, for example at least two side walls 622 and 624 positioned at an angle relative to the base 621. In some embodiments, the movable base 604 is shaped and sized to hold the robotic device while contacting at least 2, for example at least 3 adjacent external surfaces of the robotic device. Alternatively, the movable base 604 is sized and shaped to hold the robotic device while contacting only one external surface of the robotic device, optionally comprising the largest wall of the robotic device. In some embodiments, an angle between the base 621 and the side wall 622 or the side wall 624 is similar to an angle between the at least 2 external surfaces of the robotic device. In some embodiments, a surface of the movable base contacting the robotic device is sized and shaped to fit a contour of at least a portion of the robotic device. A potential advantage of holding the robotic device by contacting at least 3 adjacent surfaces and/or by fitting the base to a contour of the robotic device may be to allow steady and firm grip of the robotic device while the robotic device is activated during a surgical procedure.

### Exemplary Positioning of a Robotic Device

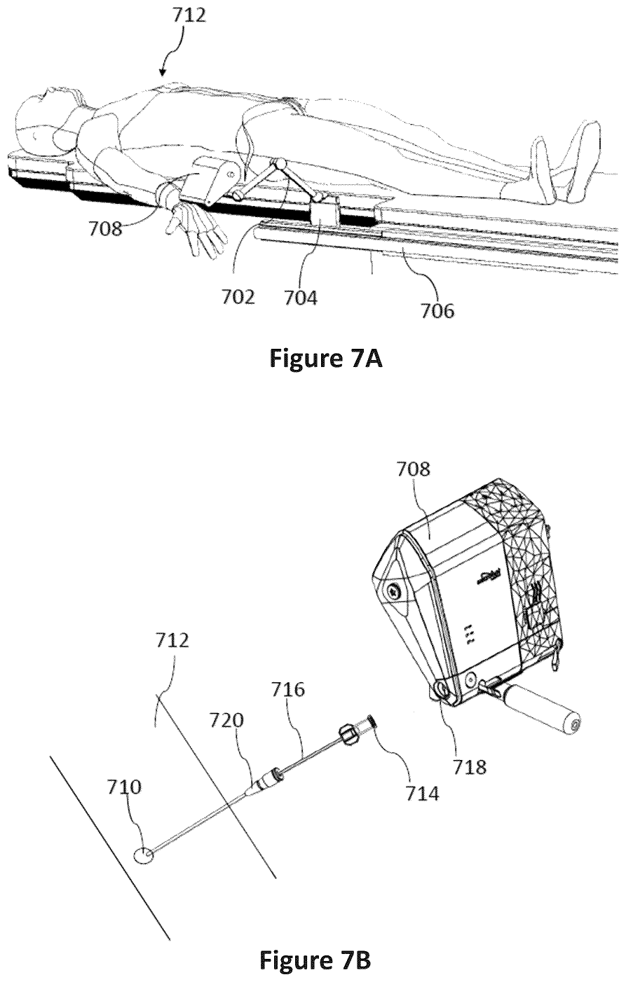

Reference is now made to FIGS. 7A-7B and FIG. 8A depicting a process for positioning a robotic device relative to a body access point, according to some exemplary embodiments of the invention.

According to some exemplary embodiments, for example as shown in FIG. 7A, an adjustable arm 702 having a distal end and a proximal end is connected by a proximal connector 704 at the proximal end to a bed 706 of a patient. In some embodiments, a robotic device 708 is attached to a distal end of the adjustable arm 702, for example to a movable base at the adjustable arm 702.

According to some exemplary embodiments, for example as shown in FIG. 7B, the adjustable arm is manipulated, for example extended and/or rotated, to position a robotic device at a distance shorter than 20 cm, for example shorter than 15 cm, shorter than 10 cm or any intermediate, smaller or larger distance from an access point 710 in a body 712 of a patient. Alternatively or additionally, the adjustable arm is manipulated, for example linearly manipulated, to position the robotic device at a distance shorter than 20 cm from a elongate surgical tool 714 extending out from the body, optionally via port 720, or from a proximal opening 714 of the elongate surgical tool 716.

Additionally, the movable base of the arm, to which the robotic device is attached, is further axially advanced, for example towards the patient body, to align an exit port 718 of the robotic device 708 with the elongate surgical tool 716 or with the elongate surgical tool proximal end 714. Alternatively or additionally, the movable base is axially advanced to position the exit port 718 at a distance shorter than 5 cm, for example shorter than 2 cm, shorter than 1 cm or any intermediate, smaller or larger distance from the elongate surgical tool 716, for example from the proximal end 714 of the elongate surgical tool.

According to some exemplary embodiments, for example as shown in FIG. 8A, the proximal opening of the elongate surgical tool 716 is then connected to the exit port 718 of the robotic device 708, to form an enclosed flow path between the robotic device and a distal opening of the elongate surgical tool positioned within the body 712. In some embodiments, the enclosed flow path is used to introduce at least one medical device, for example a surgical tool during a medical procedure, for example a surgical procedure, into the patient body, using the robotic device. In some embodiments, the at least one surgical tool comprises a catheter, a micro-catheter and/or a guidewire. Optionally, at least one actuator, for example an electric motor of the robotic device is activated in order to introduce the surgical tool into the body.

According to some exemplary embodiments, during the medical procedure, the elongate surgical tool distal end located within the body is repositioned using the robotic device, for example by axially moving the robotic device relative to the movable base of the arm. In some embodiments, the axial movement of the robotic device is limited mechanically or electrically to a range of movement of up to 10 cm, for example up to 8 cm, up to 5 cm or any intermediate, smaller or larger value.

According to some exemplary embodiments, a support member is coupled to the robotic device and to the port in the body, to prevent, for example buckling, bending and/or twisting of the elongate surgical tool, during the repositioning of the elongate surgical tool by the robotic device.

According to some exemplary embodiments, for example as shown in FIG. 8B, the adjustable arm is used to align an elongate surgical tool 716, for example a long axis of the elongate surgical tool with an inner pathway 810 of the robotic device 708. In some embodiments, the elongate surgical tool 716 is aligned with the inner pathway 810 to form an enclosed flow path between an inner pathway of the robotic device and an inner lumen of said elongate surgical tool. In some embodiments, the inner pathway 810 of the robotic device is defined by an actuation assembly 715 located within the robotic device, comprising at least two driving wheels, configured for manipulating at least one second elongate surgical tool 705. In some embodiments, the robotic device 708 comprises a second inner pathway 703 defined by actuating assembly 711, comprising at least two driving wheels. In some embodiments the second inner pathway 703 is configured to accommodate a third elongate surgical tool 701, and the actuating assembly 711 is configured to manipulate the third elongate surgical tool, for example advance/retract and/or roll.

According to some exemplary embodiments, the robotic device 708 is used to advance at least one second elongate surgical tool, for example a micro catheter 705 and/or a guide wire 701 preloaded within the robotic device 708, into the patient body via the flow path between an inner pathway 810 of the robotic device and an inner lumen of the elongate surgical tool 716, for example as described in U.S. Patent Application No. 63/195,020 filed on 30 May 2021, incorporated herein as a reference in its entirety. In some embodiments, the at least one second elongate surgical tool is introduced into the inner lumen of the elongate surgical tool 716, after the elongate surgical tool 716 is connected to the robotic device 708. Alternatively, the at least one second elongate surgical tool is introduced into the inner lumen of the elongate surgical tool 716, before connecting the elongate surgical tool 716 to the robotic device 708. In some embodiments, the robotic device 708 comprises a Y-connector, for example an embedded Y-connector 780, as further detailed and described in FIG. 8C.

Reference is now made to FIG. 8C illustrating an example for a robotic device 708 comprising an embedded Y-connector 780. In some embodiments, Y-connector 780 is provided proximally to the exit port 718. In some embodiments, the main flow path of the Y-connector 780 is axially aligned with the main flow path of the inner pathway 810, configured to be in contact with the inner lumen of an elongate surgical tool 716 connected to the robotic device via luer 714.

In some embodiments, the robotic device 708 comprises at least one actuator, for example motor 717, configured for rotating the connector located at the exit port 718, optionally via at least one gear 719.

A potential advantage of an embedded Y-connector, is that an actuator residing within the robotic device and configured for rotating the externally connected elongate surgical tool, can interface with the elongate surgical tool distally to the location of the Y-connector, enabling rotation of the tool without requiring to also rotate the Y-connector. This is in contrast to systems which do not include an embedded Y-connector, requiring a user to connect the Y-connector externally, which is located distally to the interface with a rotating actuation mechanism, causing the Y-connector to also rotate and interfere with its surroundings.

### Exemplary Body Access Points

According to some exemplary embodiments, the adjustable arm assembly is used to position the robotic device in a desired distance and/or a desired orientation relative to an access point in a patient body. In some embodiments, the access point is selected according to a surgical procedure planned to be performed in the patient, and according to the physician's discretion. Reference is now made to FIGS. 8D and 8E depicting different access points in a patient body, according to some exemplary embodiments of the invention.

According to some exemplary embodiments, for example as shown in FIG. 8D, the robotic device 708 is connected to an elongate surgical tool extending from a femoral access point 711, which allows insertion of elongate surgical tools into a femoral artery of the patient. In some embodiments, the adjustable arm is usable for positioning the robotic device in a desired position and/or orientation relative to the femoral access point 711.

According to some exemplary embodiments, for example as shown in FIG. 8E, the robotic device 708 is connected to an elongate surgical tool extending from a radial access point 713, which allows insertion of elongate surgical tool into a radial artery of the patient. In some embodiments, the adjustable arm is usable for positioning the robotic device in a desired position and/or orientation relative to the radial access point 713. In some embodiments, the adjustable arm can be used to position the robotic device in a position suitable for a carotid access point (not shown).

### Exemplary Support Member

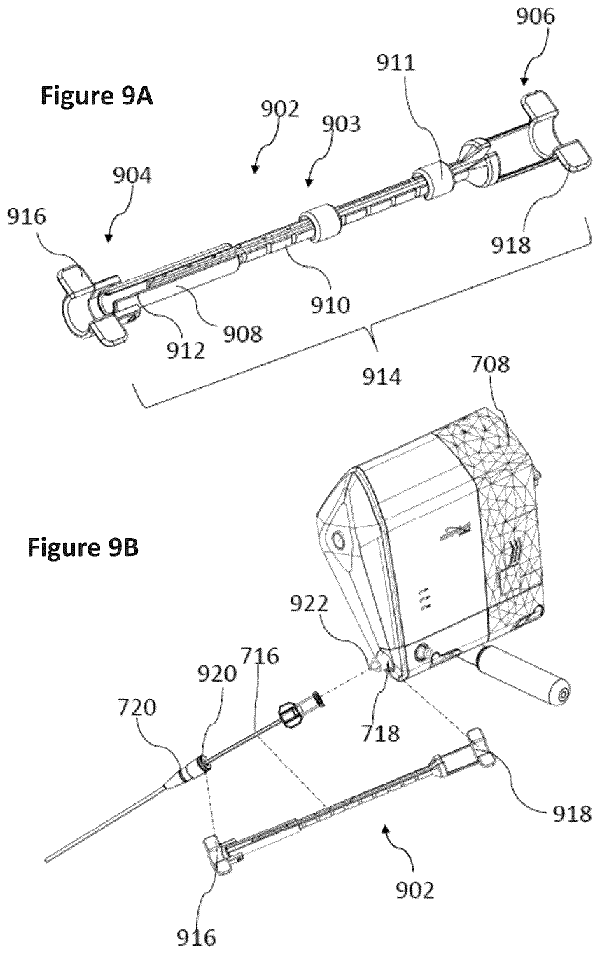

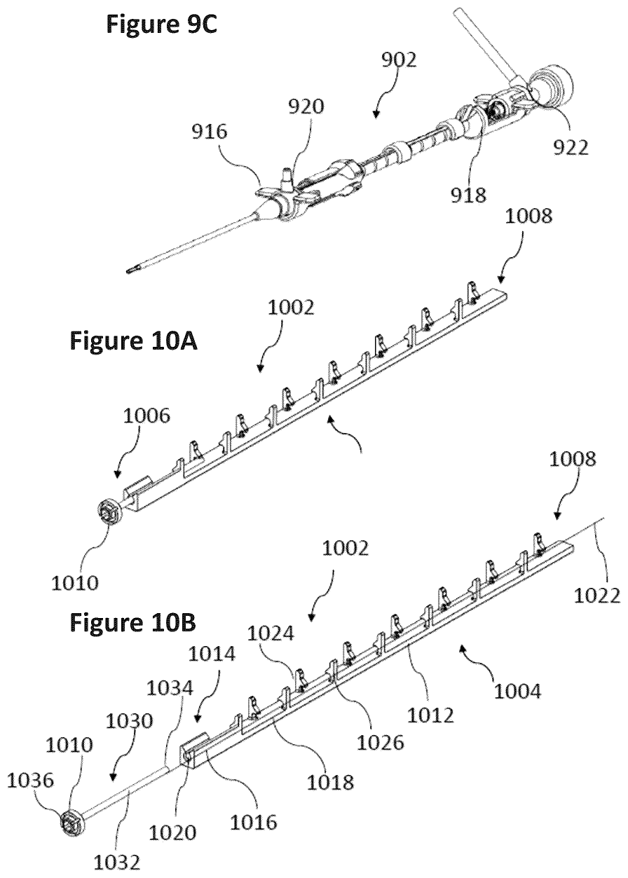

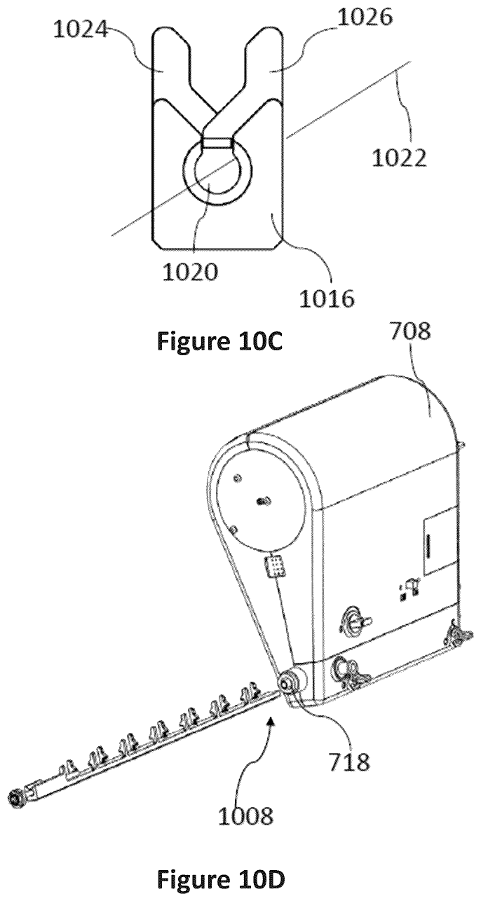

According to some exemplary embodiments, a support member is used in order to prevent buckling, bending and/or twisting of an elongate surgical tool, for example a guide catheter, extending out from a patient body. In some embodiments, the support member is coupled to a port in an access point in a patient body, for example a sheath, and to a robotic device. In some embodiments, the support member at least partly surrounds the elongate surgical tool. Reference is now made to FIGS. 9A-9C and FIGS. 10A-10D, depicting an elongate surgical tool support member, according to some exemplary embodiments of the invention.

According to some exemplary embodiments, a support member, for example support member 902 comprises a body 903 having a distal end 904 configured to be connected to a port in a body access point, and a proximal end 906 configured to be connected to a robotic device exit port, for example to a lock and/or a luer and/or a connector at the robotic device.

In some embodiments, the connector is a continuous tube having flexible characteristics which allow a certain degree of bending between the distal end 904 and the proximal end 906. In some embodiments, the connector comprises a fixed length. In some embodiments, the connector comprises an adjustable length. In some embodiments, the adjustable length is provided by a telescopic mechanism (see below). In some embodiments, the adjustable length is provided by a rotational mechanism, similar to that of a screw. In some embodiments, the adjustable length is provided by a semi-rigid helical structure incorporated within the walls of the body 903. In some embodiments, the body 903 comprises a plurality of links configured to allow the user to change the body from a straight configuration to a non-straight configuration, for example a curved configuration. In some embodiments, a potential advantage of having a plurality of links is that it allows a gradual aligning of the elongate surgical tool from the robotic device into the body access point and/or the surgical tool extending from the body access point. In some embodiments, some parts of the body comprise an adjustable length while some parts comprise a plurality of links. In some embodiments, links are configured as breaking points, to adjust the length of the body by breaking and removing any excess length.

In some embodiments, the body 903 comprises at least two movable portions, for example a first movable portion 908 and a second movable portion 910, configured to move relative to each other.

According to some exemplary embodiments, the body 903 of the support member 902 extends and/or shortens telescopically, for example by moving or sliding of the first movable portion 908 relative to the second movable portion 910, for example within, or over the second movable portion 910. In some embodiments, the first movable portion 908 slides over the second movable portion 910.

According to some exemplary embodiments, the body 903 has an inner lumen 912, formed by the first movable portion 908 and the second movable portion 910. In some embodiments, the inner lumen 912 is shaped and sized to receive an elongate surgical tool, for example a guiding catheter. In some embodiments, the inner lumen has a maximal width, for example a maximal diameter, in a range between 2 mm to 10 mm, for example between 2 mm to 6 mm, 3 mm to 8 mm, 5 mm to 10 mm, or any intermediate, smaller or larger range of values. In some embodiments, a maximal length 914 of the body 903, for example when the body 903 is fully extended, is in a range between 5 cm to 50 cm, for example in a range between 10 cm to 30 cm, or any intermediate, smaller or larger value.

According to some exemplary embodiments, the support member 902 comprises a distal connector 916 at the distal end 904, coupled to the first movable portion 908, and a proximal connector 918 at the proximal end 906, coupled to the second movable portion 910. In some embodiments, for example as shown in FIGS. 9B and 9C, the distal connector 916 is configured to be connected to a connector 920 of the sheath 720. In some embodiments, the proximal connector 918 is configured to be connected to a connector 922 at the exit port 718 of robotic device 708.

According to some exemplary embodiments, the distal connector 916 and/or the proximal connector 918 comprise at least one fastener configured to be fastened to a luer lock. In some embodiments, the at least one fastener is an elastic fastener configured to be fastened and/or snap-fitted to an external surface of the luer lock. Optionally, the at least one fastener comprises a clip, for example a flexible clip.

According to some exemplary embodiments, movement of the robotic device 708 relative to the port 720 extends or shortens, optionally telescopically, the length 914 of the body 903, while repositioning the elongate surgical tool 716 within a patient body. In some embodiments, the body 903 surrounds at least 20%, for example at least 30%, at least 40%, at least 50%, or any intermediate, smaller or larger value, of the elongate surgical tool 716 passing within the inner lumen 912. In some embodiments, each of the movable portions comprises an incomplete tube or a partially open tube having a slit along its long axis, through which at least a portion of the elongate surgical tool can be introduced. In some embodiments, the movable portions are configured to rotate one relative to the other, thereby aligning and misaligning their slits, causing the inner lumen of the incomplete tubes to open or close along the length of the overlap between the two movable portions. For example, when the two movable members are in alignment of the long axis of the slits, the inner lumen defined between the two movable members is open to receive the tool, but when one of the two movable members is rotated and its slit is misaligned with the position of the slit of the other movable member, the inner lumen is closed, at least in the portion of overlap between the two movable members. In some embodiments, at least one clip 911 is provided to enclose an open slit in at least one of the movable members, at least in portions which do not overlap with the other movable portion.

According to some exemplary embodiments, for example as shown in FIG. 9C, the connector 922 of the robotic device comprises a Y-connector.

According to some exemplary embodiments, for example as shown in FIG. 10A-10D a support member, for example a support member 1002 comprises an elongated body 1004 having a distal end 1006 and a proximal end 1008. In some embodiments, the distal end 1006 comprises a connector 1010 configured to be connected to a port. In some embodiments, the distal end 1008 is configured to be inserted into a slot in the robotic device 708 adjacent to the exit port 718, or to be coupled to a connector of the robotic device 708.

According to some exemplary embodiments, the body 1004 comprises an elongated bar 1012, optionally a flat bar or thin bar, having a distal end 1014 and the proximal end 1008, and a tubular member 1030 positioned at least partly and movable within a channel in the elongated bar 1012. In some embodiments, the bar 1012 comprises an enclosing member on a surface 1018 of the bar 1012, forming an inner channel having a distal opening 1020. In some embodiments, the enclosing member 1016 completely surrounds the inner channel. Alternatively, the enclosing member 1016 partly surrounds the inner channel, for example surrounds at least 50% of the inner channel circumference, forming, for example, a partially open channel. In some embodiments, the inner channel is aligned along a longitudinal axis 1022 of the bar 1012.

According to some exemplary embodiments, the bar 1012 comprises a plurality of protrusions, for example protrusions 1024 and 1026 extending from the surface 1018. In some embodiments, the plurality of protrusions are axially arranged on both sides of the surface 1018 and along the axis 1022, between the enclosing member 1016 and the proximal end 1008. In some embodiments, the plurality of protrusions are alternately distributed on each side of the bar 1012, along the axis 1022. In some embodiments, at least some or each of the plurality of protrusions bends or extends towards a center line of said bar 1018, aligned with the axis 1022.

According to some exemplary embodiments, the tubular member 1030 comprises a tube 1032 having a distal opening 1036 adjacent to the distal connector 1010, and a proximal opening 1034. In some embodiments, the tube 1032 is shaped and sized to receive an elongate surgical tool, and to allow free undisturbed movement of the elongate surgical tool through the tube 1032 inner lumen. In some embodiments, a minimal width of the inner lumen of tube 1032 is larger than a maximal width of the elongate surgical tool, for example to allow the free movement within the tube 1032.

According to some exemplary embodiments, the tubular member 1030 is shaped and sized to slide within the inner channel of the enclosing member 1016, and to direct a elongate surgical tool exiting from opening 1034 towards a passage formed between the plurality of the protrusions 1024 and 1026 and the surface 1018 of the elongated bar. In some embodiments, the formed passage is aligned with the axis 1022.

According to some exemplary embodiments, the support member 1002 telescopically extends and/or retracts by moving the tubular member 1030 within the enclosing member 1016 of the bar 1012.

According to some exemplary embodiments, ta length of the support member, for example support member 902 or support member 1002 is adjusted, for example according to a length of the elongate surgical tool extending between the patient body and the connector of the robotic device. In some embodiments, the length of the support member is adjusted for example by shortening of the support member, optionally by breaking or cutting the support member. In some embodiments, the support member is shortened manually. In some embodiments, the support member is cut in one or more of slits provided with the at least one portion of the support member of a plurality.

As used herein with reference to quantity or value, the term “about” means “within +10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method for positioning a robotic device connector relative to a body access point in a patient, comprising:
coupling a robotic device to a movable base located at a distal end of an adjustable arm;
manipulating the adjustable arm to align a connector of the robotic device with an elongate surgical tool extending from a body access point;
linearly moving the movable base coupled to the robotic device towards the body access point to position the connector of the robotic device in proximity to a proximal end of the elongate surgical tool;
connecting the proximal end of the elongate surgical tool to the connector of the robotic device.

2. (canceled)

3. The method according to claim 1, wherein said manipulating comprises aligning said robotic device connector with a long axis of said elongate surgical tool.

4-7. (canceled)

8. The method according to claim 1, wherein a range of movement of said adjustable arm during said manipulating is at least 2 times larger than the range of movement of said movable base during said linearly moving; and wherein said range of movement of said adjustable arm during said manipulating has at least two additional degrees of freedom compared to the degree of movement of said movable base during said linearly moving.

9-10. (canceled)

11. The method according to claim 1, comprising:
repositioning after said connecting, a distal end of the elongate surgical tool within said patient body by axially moving the robotic device relative to said movable base and along a long axis of the elongate surgical tool; wherein the repositioning is made by actuating a motor comprised within the robotic device; and wherein said repositioning comprises repositioning said elongate surgical tool while limiting an axial range of movement of said robotic device to a maximal range of movement of 10 cm.

12-14. (canceled)

15. The method according to claim 11, comprising coupling a support member to the robotic device and to a port and inserting at least a portion of the elongate surgical tool extending outside the patient body, into an inner space of the support member, thereby supporting the elongate surgical tool during said repositioning; comprising preventing buckling of said elongate surgical tool by said support member during said repositioning; and wherein the coupling comprises coupling a proximal end of the support member to the connector of the robotic device.

16. (canceled)

17. The method according to claim 15, comprising telescopically extending and/or shortening said support member during said repositioning such that prior to said coupling of the support member to the robotic device, adjusting a length of the support member in accordance with a length of the elongate surgical tool extending between the patient body and the connector of the robotic device.

18-22. (canceled)

23. The method according to claim 1, wherein said coupling of the robotic device to the movable base comprises attaching said robotic device to said movable base using at least one snap fit connector.

24. The method according to claim 1, wherein said connecting comprises connecting the proximal end of the elongate surgical tool to the connector of the robotic device to form an enclosed flow path between an inner pathway of the robotic device and an inner lumen of said elongate surgical tool.

25. The method according to claim 24, wherein said robotic device is used to advance at least one second elongate surgical tool into said patient body via said elongate surgical tool, and wherein said method further comprises loading the inner pathway of the robotic device with said at least one second elongate surgical tool prior to connecting of the proximal end of the elongate surgical tool to the connector of the robotic device.

26. A The method according to claim 25, comprising introducing a distal end of the at least one second elongate surgical tool into the inner lumen of the elongate surgical tool prior to the connecting of the proximal end of the elongate surgical tool to the connector of the robotic device; wherein said introducing comprises controllably introducing the at least one second elongate surgical tool via said inner lumen into said patient body using at least one second actuator of the robotic device; and wherein said controllably introducing is performed during a medical procedure, and wherein said method further comprises discarding said robotic device, and/or said adjustable arm after completing said medical procedure.

27-33. (canceled)

34. A maneuverable arm assembly, comprising:
an adjustable arm configured to move in at least 3 degrees of freedom, having a distal end and a proximal end, comprising a lock configured to lock said adjustable arm in a specific orientation and/or position relative to an access point in a patient body;
a movable base coupled to said distal end of said adjustable arm, wherein said movable base is configured to linearly move relative to said distal end, wherein said movable base comprises at least one fastener for coupling with a coupler of a robotic device;
wherein said at least one fastener of said movable base is configured to reversibly couple with said robotic device coupler by an adaptor having a first end coupled to said robotic device and a second end coupled to said movable base.

35. The arm assembly according to claim 34, wherein said movable base axially slides to a maximal distance of up to 10 cm from said distal end; and wherein a maximal extension distance of said adjustable arm between said distal end and said proximal end is in a range between 30 cm to 3 meters.

36-37. (canceled)

38. The arm assembly according to claim 34, wherein said at least one fastener comprises a snap-fit fastener; wherein said snap-fit fastener in said movable base comprises a protruding flexible edge shaped and sized to interlock with a snap-in region of a robotic device; and wherein said snap-fit fastener comprising a pushable lever extending out from said movable base and functionally coupled to said protruding flexible edge, wherein pushing said pushable lever releases said protruding flexible edge from said snap-in region.

39-40. (canceled)

41. The arm assembly according to claim 34, wherein said adjustable arm comprises a torque limiting knob, wherein rotation of said torque limiting knob locks said adjustable arm with a limited predetermined locking force; wherein the distal end of said adjustable arm comprises a distal connector coupled to said distal end via a ball joint, and wherein said distal connector comprises a slot, and wherein said movable base is coupled to said distal connector and axially slides within said slot.

42-43. (canceled)

44. The arm assembly according to claim 41, wherein said movable base further comprises a lock for locking said movable base at a desired position within said slot; and a rotating handle with said lock, wherein rotation of said handle locks said movable base in said slot.

45. (canceled)

46. The arm assembly according to claim 41, wherein said adjustable arm is a multi-joint arm comprising at least one first elongated portion and at least one second elongated portion pivotally connected to each other by at least one hinge passing therebetween in a central hinge portion comprising said torque limiting knob; and wherein said adjustable arm further comprises a proximal connector coupled to said proximal end via a ball joint, and configured to couple said adjustable arm to an anchoring point.

47-53. (canceled)

54. The arm assembly according to claim 34, wherein said first end of said adaptor is positioned within a slot in said robotic device, and is functionally coupled to at least one actuator of said robotic device, wherein said at least one actuator is configured to axially move said robotic device relative to said movable base by moving said adaptor within said robotic device slot.

55-56. (canceled)

57. The arm assembly according to claim 34, comprising a telescopic support member having an inner passage along a longitudinal axis of said telescopic support member, a proximal end with a proximal connector configured to couple said proximal end to said robotic device near at least one exit port, and a distal end with a distal connector configured to couple said distal end to a access point, wherein said inner passage is shaped and sized to receive an elongate surgical tool extending from said access point.

58-59. (canceled)

60. The arm assembly according to claim 57, wherein said telescopic support member comprises at least one first movable portion and at least one second movable portion slidable within said at least one first movable portion, and wherein said at least one first movable portion and said at least one a second movable portion define said inner passage.

61. The arm assembly according to claim 54, wherein activation of said at least one actuator of said robotic device axially moves said robotic device relative to said movable base with telescopically extending or shortening said telescopic support member.

62-71. (canceled)
